SEQUENCE LISTING (45 sequences)

SEQ ID NO: 1 - DNA, 1363 bp, Glycine max
cccgggaagtgagttatttatgtttataagtggatttgtatatggaatgtgacacataatgagagttttactttgtcttggagcagtaatgtcatgccttttctgcatacttggaaaggtggcacacatgcacgatatgaaggtttaggttgcttccacgattgctaggcgttgtctatttgcatgttcttctgcatggtattaagaagttcttagagaattaatctaagtacattttttttggtctggatcagacatcatatggatgctttcaaattcatgcgttggagattaattttactcataataggtaattatattaattaaaagaaattttacataaaaatacaacataaattattccattaaatatattattccctgtgactacaatgagataatctaagtgtatttgaaagtggaacagtagaaattataaaaattgcaatgagttgaataaaaaaggttggattaagaaagtaatctaagtacatttggaagtggaatagtagaaataaaattaaatgagttgaaattgaaaataattaaaaaaagtagggctaagaaatttctccttcaacttcatgatagcaaatattccattaggccatttgtagtttatgaatgagtatatataatcatgattttaggaattcgatctgctcgacacaaccgtgttacactttttttaaaatgtcatcataaaaataaaaaataaaagacatgttataattaagaataaggtgatcagtataaaaataagtaattttgggaaatattaaagttcaaaaaagaactattgaaagaaagaatattattatttaaaaagagaaaagaaaatgatgaaatgctattttcagttaaagaaaataagaaaaaaaaatacaaagaataattcaatgctggggctgtatatatgtttaagatgataattttttttttttttaaaaaaagataagaattaaatattttctcctttaatttctgaatcacggttttggttctgataagacactgattagtcacccatcaaatataatgaactaattctcctattctatttcaaaattttgattatacttagattaattttctaatatacttggacctgtttttcatgcagaagatgcagatatagctagacagcacctagtaatcgtggaaccaacaccaatgtccatatcatgcatgtgtgccacctttcaaatgtaatccagtagtaaaaaaagccatgacatgtaactccacgacagagtaaaactctcagaagtacctctcgtttcatatctgcaaatcctctaatataaataactcacttcacgggttcttttctcttcacagcaaaaacaattaataaccatgg

SEQ ID NO: 2 - DNA, 1116 bp, Glycine max
ggtctggatcagacatcatatggatgctttcaaattcatgcgttggagattaattttactcataataggtaattatattaattaaaagaaattttacataaaaatacaacataaattattccattaaatatattattccctgtgactacaatgagataatctaagtgtatttgaaagtggaacagtagaaattataaaaattgcaatgagttgaataaaaaaggttggattaagaaagtaatctaagtacatttggaagtggaatagtagaaataaaattaaatgagttgaaattgaaaataattaaaaaaagtagggctaagaaatttctccttcaacttcatgatagcaaatattccattaggccatttgtagtttatgaatgagtatatataatcatgattttaggaattcgatctgctcgacacaaccgtgttacactttttttaaaatgtcatcataaaaataaaaaataaaagacatgttataattaagaataaggtgatcagtataaaaataagtaattttgggaaatattaaagttcaaaaaagaactattgaaagaaagaatattattatttaaaaagagaaaagaaaatgatgaaatgctattttcagttaaagaaaataagaaaaaaaaatacaaagaataattcaatgctggggctgtatatatgtttaagatgataattttttttttttttaaaaaaagataagaattaaatattttctcctttaatttctgaatcacggttttggttctgataagacactgattagtcacccatcaaatataatgaactaattctcctattctatttcaaaattttgattatacttagattaattttctaatatacttggacctgtttttcatgcagaagatgcagatatagctagacagcacctagtaatcgtggaaccaacaccaatgtccatatcatgcatgtgtgccacctttcaaatgtaatccagtagtaaaaaaagccatgacatgtaactccacgacagagtaaaactctcagaagtacctctcgtttcatatctgcaaatcctctaatataaataactcacttcacgggttcttttctcttcacagcaaaaacaattaataac

SEQ ID NO: 3 - DNA, 885 bp, Glycine max
aagaaagtaatctaagtacatttggaagtggaatagtagaaataaaattaaatgagttgaaattgaaaataattaaaaaaagtagggctaagaaatttctccttcaacttcatgatagcaaatattccattaggccatttgtagtttatgaatgagtatatataatcatgattttaggaattcgatctgctcgacacaaccgtgttacactttttttaaaatgtcatcataaaaataaaaaataaaagacatgttataattaagaataaggtgatcagtataaaaataagtaattttgggaaatattaaagttcaaaaaagaactattgaaagaaagaatattattatttaaaaagagaaaagaaaatgatgaaatgctattttcagttaaagaaaataagaaaaaaaaatacaaagaataattcaatgctggggctgtatatatgtttaagatgataattttttttttttttaaaaaaagataagaattaaatattttctcctttaatttctgaatcacggttttggttctgataagacactgattagtcacccatcaaatataatgaactaattctcctattctatttcaaaattttgattatacttagattaattttctaatatacttggacctgtttttcatgcagaagatgcagatatagctagacagcacctagtaatcgtggaaccaacaccaatgtccatatcatgcatgtgtgccacctttcaaatgtaatccagtagtaaaaaaagccatgacatgtaactccacgacagagtaaaactctcagaagtacctctcgtttcatatctgcaaatcctctaatataaataactcacttcacgggttcttttctcttcacagcaaaaacaattaataac

SEQ ID NO: 4 - DNA, 698 bp, Glycine max
tgctcgacacaaccgtgttacactttttttaaaatgtcatcataaaaataaaaaataaaagacatgttataattaagaataaggtgatcagtataaaaataagtaattttgggaaatattaaagttcaaaaaagaactattgaaagaaagaatattattatttaaaaagagaaaagaaaatgatgaaatgctattttcagttaaagaaaataagaaaaaaaaatacaaagaataattcaatgctggggctgtatatatgtttaagatgataattttttttttttttaaaaaaagataagaattaaatattttctcctttaatttctgaatcacggttttggttctgataagacactgattagtcacccatcaaatataatgaactaattctcctattctatttcaaaattttgattatacttagattaattttctaatatacttggacctgtttttcatgcagaagatgcagatatagctagacagcacctagtaatcgtggaaccaacaccaatgtccatatcatgcatgtgtgccacctttcaaatgtaatccagtagtaaaaaaagccatgacatgtaactccacgacagagtaaaactctcagaagtacctctcgtttcatatctgcaaatcctctaatataaataactcacttcacgggttcttttctcttcacagcaaaaacaattaataac

SEQ ID NO: 5 - DNA, 473 bp, Glycine max
caaagaataattcaatgctggggctgtatatatgtttaagatgataattttttttttttttaaaaaaagataagaattaaatattttctcctttaatttctgaatcacggttttggttctgataagacactgattagtcacccatcaaatataatgaactaattctcctattctatttcaaaattttgattatacttagattaattttctaatatacttggacctgtttttcatgcagaagatgcagatatagctagacagcacctagtaatcgtggaaccaacaccaatgtccatatcatgcatgtgtgccacctttcaaatgtaatccagtagtaaaaaaagccatgacatgtaactccacgacagagtaaaactctcagaagtacctctcgtttcatatctgcaaatcctctaatataaataactcacttcacgggttcttttctcttcacagcaaaaacaattaataac

SEQ ID NO: 6 - DNA, 245 bp, Glycine max
tttcatgcagaagatgcagatatagctagacagcacctagtaatcgtggaaccaacaccaatgtccatatcatgcatgtgtgccacctttcaaatgtaatccagtagtaaaaaaagccatgacatgtaactccacgacagagtaaaactctcagaagtacctctcgtttcatatctgcaaatcctctaatataaataactcacttcacgggttcttttctcttcacagcaaaaacaattaataac

SEQ ID NO: 7 - DNA, 32 bp, Artificial sequence
  primer, PSO333255Xma
cccgggaagtgagttatttatgtttataagtg

SEQ ID NO: 8 - DNA, 40 bp, Artificial sequence
  primer, PSO333255Nco
ccatggttattaattgtttttgctgtgaagagaaaagaac

SEQ ID NO: 9 - DNA, 32 bp, Artificial sequence
  primer, QC489-A
gttattaattgtttttgctgtgaagagaaaag

SEQ ID NO: 10 - DNA, 26 bp, Artificial sequence
  primer, QC489-S1
ggtctggatcagacatcatatggatg

SEQ ID NO: 11 - DNA, 31 bp, Artificial sequence
  primer, QC489-S2
aagaaagtaatctaagtacatttggaagtgg

SEQ ID NO: 12 - DNA, 23 bp, Artificial sequence
  primer, QC489-S3
tgctcgacacaaccgtgttacac

SEQ ID NO: 13 - DNA, 24 bp, Artificial sequence
  primer, QC489-S4
caaagaataattcaatgctggggc

SEQ ID NO: 14 - DNA, 26 bp, Artificial sequence
  primer, QC489-S5
tttcatgcagaagatgcagatatagc

SEQ ID NO: 15 - DNA, 482 bp, Glycine max
acagcaaaaacaattaataaagatgagtttgaagaacaacatggtggtgctaaaggtgtgtttgttgcttcttttccttgtgggggttacagctgcacgcatggaactgagcttcttcaaaagtgatcagtcatcaagttatgatgatgatgagtattcaaaaccatgctgtgatctctgcatgtgcacacgctcaatgcctcctcaatgcagctgtgaagatattaggctgaattcatgccactcagattgtaagagctgtatgtgcacacgctcacagccaggacagtgtcgttgtcttgacaccaacgacttctgctacaaaccttgcaagtccagagatgactagaaaaactaatagctctctcaaatggacgaagcccctttaggctttgtttgttatgttaggggagacaaataaaacaagaaataaaagctcagtggccagtaatttgcttttagcaaatttggtcatttttatt

SEQ ID NO: 16 - protein, 108 aa, Glycine max
MSLKNNMVVLKVCLLLLFLVGVTAARMELSFFKSDQSSSYDDDEYSKPCCDLCMCTRSMPPQCSCEDIRLNSCHSDCKSCMCTRSQPGQCRCLDTNDFCYKPCKSRDD

SEQ ID NO: 17 - DNA, 4640 bp, Artificial sequence
  QC489
ccgggaagtgagttatttatgtttataagtggatttgtatatggaatgtgacacataatgagagttttactttgtcttggagcagtaatgtcatgccttttctgcatacttggaaaggtggcacacatgcacgatatgaaggtttaggttgcttccacgattgctaggcgttgtctatttgcatgttcttctgcatggtattaagaagttcttagagaattaatctaagtacattttttttggtctggatcagacatcatatggatgctttcaaattcatgcgttggagattaattttactcataataggtaattatattaattaaaagaaattttacataaaaatacaacataaattattccattaaatatattattccctgtgactacaatgagataatctaagtgtatttgaaagtggaacagtagaaattataaaaattgcaatgagttgaataaaaaaggttggattaagaaagtaatctaagtacatttggaagtggaatagtagaaataaaattaaatgagttgaaattgaaaataattaaaaaaagtagggctaagaaatttctccttcaacttcatgatagcaaatattccattaggccatttgtagtttatgaatgagtatatataatcatgattttaggaattcgatctgctcgacacaaccgtgttacactttttttaaaatgtcatcataaaaataaaaaataaaagacatgttataattaagaataaggtgatcagtataaaaataagtaattttgggaaatattaaagttcaaaaaagaactattgaaagaaagaatattattatttaaaaagagaaaagaaaatgatgaaatgctattttcagttaaagaaaataagaaaaaaaaatacaaagaataattcaatgctggggctgtatatatgtttaagatgataattttttttttttttaaaaaaagataagaattaaatattttctcctttaatttctgaatcacggttttggttctgataagacactgattagtcacccatcaaatataatgaactaattctcctattctatttcaaaattttgattatacttagattaattttctaatatacttggacctgtttttcatgcagaagatgcagatatagctagacagcacctagtaatcgtggaaccaacaccaatgtccatatcatgcatgtgtgccacctttcaaatgtaatccagtagtaaaaaaagccatgacatgtaactccacgacagagtaaaactctcagaagtacctctcgtttcatatctgcaaatcctctaatataaataactcacttcacgggttcttttctcttcacagcaaaaacaattaataaccatggcccacagcaagcacggcctgaaggaggagatgaccatgaagtaccacatggagggctgcgtgaacggccacaagttcgtgatcaccggcgagggcatcggctaccccttcaagggcaagcagaccatcaacctgtgcgtgatcgagggcggccccctgcccttcagcgaggacatcctgagcgccggcttcaagtacggcgaccggatcttcaccgagtacccccaggacatcgtggactacttcaagaacagctgccccgccggctacacctggggccggagcttcctgttcgaggacggcgccgtgtgcatctgtaacgtggacatcaccgtgagcgtgaaggagaactgcatctaccacaagagcatcttcaacggcgtgaacttccccgccgacggccccgtgatgaagaagatgaccaccaactgggaggccagctgcgagaagatcatgcccgtgcctaagcagggcatcctgaagggcgacgtgagcatgtacctgctgctgaaggacggcggccggtaccggtgccagttcgacaccgtgtacaaggccaagagcgtgcccagcaagatgcccgagtggcacttcatccagcacaagctgctgcgggaggaccggagcgacgccaagaaccagaagtggcagctgaccgagcacgccatcgccttccccagcgccctggcctgagagctcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattctagtggccggcccagctgatatccatcacactggcggccgcactcgactgaattggttccggcgccagcctgcttttttgtacaaagttggcattataaaaaagcattgcttatcaatttgttgcaacgaacaggtcactatcagtcaaaataaaatcattatttggggcccgagcttaagtaactaactaacaggaagagtttgtagaaacgcaaaaaggccatccgtcaggatggccttctgcttagtttgatgcctggcagtttatggcgggcgtcctgcccgccaccctccgggccgttgcttcacaacgttcaaatccgctcccggcggatttgtcctactcaggagagcgttcaccgacaaacaacagataaaacgaaaggcccagtcttccgactgagcctttcgttttatttgatgcctggcagttccctactctcgcttagtagttagacgtccccgagatccatgctagcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacggggcccaatctgaataatgttacaaccaattaaccaattctgattagaaaaactcatcgagcatcaaatgaaactgcaatttattcatatcaggattatcaataccatatttttgaaaaagccgtttctgtaatgaaggagaaaactcaccgaggcagttccataggatggcaagatcctggtatcggtctgcgattccgactcgtccaacatcaatacaacctattaatttcccctcgtcaaaaataaggttatcaagtgagaaatcaccatgagtgacgactgaatccggtgagaatggcaaaagtttatgcatttctttccagacttgttcaacaggccagccattacgctcgtcatcaaaatcactcgcatcaaccaaaccgttattcattcgtgattgcgcctgagcgagacgaaatacgcgatcgctgttaaaaggacaattacaaacaggaatcgaatgcaaccggcgcaggaacactgccagcgcatcaacaatattttcacctgaatcaggatattcttctaatacctggaatgctgtttttccggggatcgcagtggtgagtaaccatgcatcatcaggagtacggataaaatgcttgatggtcggaagaggcataaattccgtcagccagtttagtctgaccatctcatctgtaacatcattggcaacgctacctttgccatgtttcagaaacaactctggcgcatcgggcttcccatacaagcgatagattgtcgcacctgattgcccgacattatcgcgagcccatttatacccatataaatcagcatccatgttggaatttaatcgcggcctcgacgtttcccgttgaatatggctcataacaccccttgtattactgtttatgtaagcagacagttttattgttcatgatgatatatttttatcttgtgcaatgtaacatcagagattttgagacacgggccagagctgcagctggatggcaaataatgattttattttgactgatagtgacctgttcgttgcaacaaattgataagcaatgctttcttataatgccaactttgtacaagaaagctgggtctagatatctcgac

SEQ ID NO: 18 - DNA, 8482 bp, Artificial sequence
  QC478i
atcgaaccactttgtacaagaaagctgaacgagaaacgtaaaatgatataaatatcaatatattaaattagattttgcataaaaaacagactacataatactgtaaaacacaacatatccagtcactatggtcgacctgcagactggctgtgtataagggagcctgacatttatattccccagaacatcaggttaatggcgtttttgatgtcattttcgcggtggctgagatcagccacttcttccccgataacggagaccggcacactggccatatcggtggtcatcatgcgccagctttcatccccgatatgcaccaccgggtaaagttcacgggggactttatctgacagcagacgtgcactggccagggggatcaccatccgtcgcccgggcgtgtcaataatatcactctgtacatccacaaacagacgataacggctctctcttttataggtgtaaaccttaaactgcatttcaccagcccctgttctcgtcagcaaaagagccgttcatttcaataaaccgggcgacctcagccatcccttcctgattttccgctttccagcgttcggcacgcagacgacgggcttcattctgcatggttgtgcttaccagaccggagatattgacatcatatatgccttgagcaactgatagctgtcgctgtcaactgtcactgtaatacgctgcttcatagcatacctctttttgacatacttcgggtatacatatcagtatatattcttataccgcaaaaatcagcgcgcaaatacgcatactgttatctggcttttagtaagccggatcctctagattacgccccgcctgccactcatcgcagtactgttgtaattcattaagcattctgccgacatggaagccatcacaaacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacggaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgacggatcctaactcaaaatccacacattatacgagccggaagcataaagtgtaaagcctggggtgcctaatgcggccgccatagtgactggatatgttgtgttttacagtattatgtagtctgttttttatgcaaaatctaatttaatatattgatatttatatcattttacgtttctcgttcagcttttttgtacaaacttgtttgataaacactagtaacggccgccagtgtgctggaattcgcccttcccaagctttgctctagatcaaactcacatccaaacataacatggatatcttccttaccaatcatactaattattttgggttaaatattaatcattatttttaagatattaattaagaaattaaaagattttttaaaaaaatgtataaaattatattattcatgatttttcatacatttgattttgataataaatatattttttttaatttcttaaaaaatgttgcaagacacttattagacatagtcttgttctgtttacaaaagcattcatcatttaatacattaaaaaatatttaatactaacagtagaatcttcttgtgagtggtgtgggagtaggcaacctggcattgaaacgagagaaagagagtcagaaccagaagacaaataaaaagtatgcaacaaacaaatcaaaatcaaagggcaaaggctggggttggctcaattggttgctacattcaattttcaactcagtcaacggttgagattcactctgacttccccaatctaagccgcggatgcaaacggttgaatctaacccacaatccaatctcgttacttaggggcttttccgtcattaactcacccctgccacccggtttccctataaattggaactcaatgctcccctctaaactcgtatcgcttcagagttgagaccaagacacactcgttcatatatctctctgctcttctcttctcttctacctctcaaggtacttttcttctccctctaccaaatcctagattccgtggttcaatttcggatcttgcacttctggtttgctttgccttgctttttcctcaactgggtccatctaggatccatgtgaaactctactctttctttaatatctgcggaatacgcgtttgactttcagatctagtcgaaatcatttcataattgcctttctttcttttagcttatgagaaataaaatcactttttttttatttcaaaataaaccttgggccttgtgctgactgagatggggtttggtgattacagaattttagcgaattttgtaattgtacttgtttgtctgtagttttgttttgttttcttgtttctcatacattccttaggcttcaattttattcgagtataggtcacaataggaattcaaactttgagcaggggaattaatcccttccttcaaatccagtttgtttgtatatatgtttaaaaaatgaaacttttgctttaaattctattataactttttttatggctgaaatttttgcatgtgtctttgctctctgttgtaaatttactgtttaggtactaactctaggcttgttgtgcagtttttgaagtataacaacagaagttcctattccgaagttcctattctctagaaagtataggaacttccaccacacaacacaatggcggccaccgcttccagaaccacccgattctcttcttcctcttcacaccccaccttccccaaacgcattactagatccaccctccctctctctcatcaaaccctcaccaaacccaaccacgctctcaaaatcaaatgttccatctccaaaccccccacggcggcgcccttcaccaaggaagcgccgaccacggagcccttcgtgtcacggttcgcctccggcgaacctcgcaagggcgcggacatccttgtggaggcgctggagaggcagggcgtgacgacggtgttcgcgtaccccggcggtgcgtcgatggagatccaccaggcgctcacgcgctccgccgccatccgcaacgtgctcccgcgccacgagcagggcggcgtcttcgccgccgaaggctacgcgcgttcctccggcctccccggcgtctgcattgccacctccggccccggcgccaccaacctcgtgagcggcctcgccgacgctttaatggacagcgtcccagtcgtcgccatcaccggccaggtcgcccgccggatgatcggcaccgacgccttccaagaaaccccgatcgtggaggtgagcagatccatcacgaagcacaactacctcatcctcgacgtcgacgacatcccccgcgtcgtcgccgaggctttcttcgtcgccacctccggccgccccggtccggtcctcatcgacattcccaaagacgttcagcagcaactcgccgtgcctaattgggacgagcccgttaacctccccggttacctcgccaggctgcccaggccccccgccgaggcccaattggaacacattgtcagactcatcatggaggcccaaaagcccgttctctacgtcggcggtggcagtttgaattccagtgctgaattgaggcgctttgttgaactcactggtattcccgttgctagcactttaatgggtcttggaacttttcctattggtgatgaatattcccttcagatgctgggtatgcatggtactgtttatgctaactatgctgttgacaatagtgatttgttgcttgcctttggggtaaggtttgatgaccgtgttactgggaagcttgaggcttttgctagtagggctaagattgttcacattgatattgattctgccgagattgggaagaacaagcaggcgcacgtgtcggtttgcgcggatttgaagttggccttgaagggaattaatatgattttggaggagaaaggagtggagggtaagtttgatcttggaggttggagagaagagattaatgtgcagaaacacaagtttccattgggttacaagacattccaggacgcgatttctccgcagcatgctatcgaggttcttgatgagttgactaatggagatgctattgttagtactggggttgggcagcatcaaatgtgggctgcgcagttttacaagtacaagagaccgaggcagtggttgacctcagggggtcttggagccatgggttttggattgcctgcggctattggtgctgctgttgctaaccctggggctgttgtggttgacattgatggggatggtagtttcatcatgaatgttcaggagttggccactataagagtggagaatctcccagttaagatattgttgttgaacaatcagcatttgggtatggtggttcagttggaggataggttctacaagtccaatagagctcacacctatcttggagatccgtctagcgagagcgagatattcccaaacatgctcaagtttgctgatgcttgtgggataccggcagcgcgagtgacgaagaaggaagagcttagagcggcaattcagagaatgttggacacccctggcccctaccttcttgatgtcattgtgccccatcaggagcatgtgttgccgatgattcccagtaatggatccttcaaggatgtgataactgagggtgatggtagaacgaggtactgattgcctagaccaaatgttccttgatgcttgttttgtacaatatatataagataatgctgtcctagttgcaggatttggcctgtggtgagcatcatagtctgtagtagttttggtagcaagacattttattttccttttatttaacttactacatgcagtagcatctatctatctctgtagtctgatatctcctgttgtctgtattgtgccgttggattttttgctgtagtgagactgaaaatgatgtgctagtaataatatttctgttagaaatctaagtagagaatctgttgaagaagtcaaaagctaatggaatcaggttacatattcaatgtttttctttttttagcggttggtagacgtgtagattcaacttctcttggagctcacctaggcaatcagtaaaatgcatattccttttttaacttgccatttatttacttttagtggaaattgtgaccaatttgttcatgtagaacggatttggaccattgcgtccacaaaacgtctcttttgctcgatcttcacaaagcgataccgaaatccagagatagttttcaaaagtcagaaatggcaaagttataaatagtaaaacagaatagatgctgtaatcgacttcaataacaagtggcatcacgtttctagttctagacccatcagctgggccggcccagctgatgatcccggtgaagttcctattccgaagttcctattctccagaaagtataggaacttcactagagcttgcggccgcgcatgctgacttaatcagctaacgccactcgagggggggcccggtaccggcgcgccgttctatagtgtcacctaaatcgtatgtgtatgatacataaggttatgtattaattgtagccgcgttctaacgacaatatgtccatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcaggttgatcagatctcgatcccgcgaaattaatacgactcactatagggagaccacaacggtttccctctagaaataattttgtttaactttaagaaggagatatacccatggaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggctatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagtgaggtacagcttggatcgatccggctgctaacaaagcccgaaaggaagctgagttggctgctgccaccgctgagcaataactagcataaccccttggggcctctaaacgggtcttgaggggttttttgctgaaaggaggaactatatccggatgctcgggcgcgccggtacccgggtaccgagctcactagacgcggtgaaattacctaattaacaccggtgttt

SEQ ID NO: 19 - DNA, 9239 bp, Artificial sequence
  QC607
ccgggaagtgagttatttatgtttataagtggatttgtatatggaatgtgacacataatgagagttttactttgtcttggagcagtaatgtcatgccttttctgcatacttggaaaggtggcacacatgcacgatatgaaggtttaggttgcttccacgattgctaggcgttgtctatttgcatgttcttctgcatggtattaagaagttcttagagaattaatctaagtacattttttttggtctggatcagacatcatatggatgctttcaaattcatgcgttggagattaattttactcataataggtaattatattaattaaaagaaattttacataaaaatacaacataaattattccattaaatatattattccctgtgactacaatgagataatctaagtgtatttgaaagtggaacagtagaaattataaaaattgcaatgagttgaataaaaaaggttggattaagaaagtaatctaagtacatttggaagtggaatagtagaaataaaattaaatgagttgaaattgaaaataattaaaaaaagtagggctaagaaatttctccttcaacttcatgatagcaaatattccattaggccatttgtagtttatgaatgagtatatataatcatgattttaggaattcgatctgctcgacacaaccgtgttacactttttttaaaatgtcatcataaaaataaaaaataaaagacatgttataattaagaataaggtgatcagtataaaaataagtaattttgggaaatattaaagttcaaaaaagaactattgaaagaaagaatattattatttaaaaagagaaaagaaaatgatgaaatgctattttcagttaaagaaaataagaaaaaaaaatacaaagaataattcaatgctggggctgtatatatgtttaagatgataattttttttttttttaaaaaaagataagaattaaatattttctcctttaatttctgaatcacggttttggttctgataagacactgattagtcacccatcaaatataatgaactaattctcctattctatttcaaaattttgattatacttagattaattttctaatatacttggacctgtttttcatgcagaagatgcagatatagctagacagcacctagtaatcgtggaaccaacaccaatgtccatatcatgcatgtgtgccacctttcaaatgtaatccagtagtaaaaaaagccatgacatgtaactccacgacagagtaaaactctcagaagtacctctcgtttcatatctgcaaatcctctaatataaataactcacttcacgggttcttttctcttcacagcaaaaacaattaataaccatggcccacagcaagcacggcctgaaggaggagatgaccatgaagtaccacatggagggctgcgtgaacggccacaagttcgtgatcaccggcgagggcatcggctaccccttcaagggcaagcagaccatcaacctgtgcgtgatcgagggcggccccctgcccttcagcgaggacatcctgagcgccggcttcaagtacggcgaccggatcttcaccgagtacccccaggacatcgtggactacttcaagaacagctgccccgccggctacacctggggccggagcttcctgttcgaggacggcgccgtgtgcatctgtaacgtggacatcaccgtgagcgtgaaggagaactgcatctaccacaagagcatcttcaacggcgtgaacttccccgccgacggccccgtgatgaagaagatgaccaccaactgggaggccagctgcgagaagatcatgcccgtgcctaagcagggcatcctgaagggcgacgtgagcatgtacctgctgctgaaggacggcggccggtaccggtgccagttcgacaccgtgtacaaggccaagagcgtgcccagcaagatgcccgagtggcacttcatccagcacaagctgctgcgggaggaccggagcgacgccaagaaccagaagtggcagctgaccgagcacgccatcgccttccccagcgccctggcctgagagctcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattctagtggccggcccagctgatatccatcacactggcggccgcactcgactgaattggttccggcgccagcctgcttttttgtacaaacttgtttgataaacactagtaacggccgccagtgtgctggaattcgcccttcccaagctttgctctagatcaaactcacatccaaacataacatggatatcttccttaccaatcatactaattattttgggttaaatattaatcattatttttaagatattaattaagaaattaaaagattttttaaaaaaatgtataaaattatattattcatgatttttcatacatttgattttgataataaatatattttttttaatttcttaaaaaatgttgcaagacacttattagacatagtcttgttctgtttacaaaagcattcatcatttaatacattaaaaaatatttaatactaacagtagaatcttcttgtgagtggtgtgggagtaggcaacctggcattgaaacgagagaaagagagtcagaaccagaagacaaataaaaagtatgcaacaaacaaatcaaaatcaaagggcaaaggctggggttggctcaattggttgctacattcaattttcaactcagtcaacggttgagattcactctgacttccccaatctaagccgcggatgcaaacggttgaatctaacccacaatccaatctcgttacttaggggcttttccgtcattaactcacccctgccacccggtttccctataaattggaactcaatgctcccctctaaactcgtatcgcttcagagttgagaccaagacacactcgttcatatatctctctgctcttctcttctcttctacctctcaaggtacttttcttctccctctaccaaatcctagattccgtggttcaatttcggatcttgcacttctggtttgctttgccttgctttttcctcaactgggtccatctaggatccatgtgaaactctactctttctttaatatctgcggaatacgcgtttgactttcagatctagtcgaaatcatttcataattgcctttctttcttttagcttatgagaaataaaatcactttttttttatttcaaaataaaccttgggccttgtgctgactgagatggggtttggtgattacagaattttagcgaattttgtaattgtacttgtttgtctgtagttttgttttgttttcttgtttctcatacattccttaggcttcaattttattcgagtataggtcacaataggaattcaaactttgagcaggggaattaatcccttccttcaaatccagtttgtttgtatatatgtttaaaaaatgaaacttttgctttaaattctattataactttttttatggctgaaatttttgcatgtgtctttgctctctgttgtaaatttactgtttaggtactaactctaggcttgttgtgcagtttttgaagtataacaacagaagttcctattccgaagttcctattctctagaaagtataggaacttccaccacacaacacaatggcggccaccgcttccagaaccacccgattctcttcttcctcttcacaccccaccttccccaaacgcattactagatccaccctccctctctctcatcaaaccctcaccaaacccaaccacgctctcaaaatcaaatgttccatctccaaaccccccacggcggcgcccttcaccaaggaagcgccgaccacggagcccttcgtgtcacggttcgcctccggcgaacctcgcaagggcgcggacatccttgtggaggcgctggagaggcagggcgtgacgacggtgttcgcgtaccccggcggtgcgtcgatggagatccaccaggcgctcacgcgctccgccgccatccgcaacgtgctcccgcgccacgagcagggcggcgtcttcgccgccgaaggctacgcgcgttcctccggcctccccggcgtctgcattgccacctccggccccggcgccaccaacctcgtgagcggcctcgccgacgctttaatggacagcgtcccagtcgtcgccatcaccggccaggtcgcccgccggatgatcggcaccgacgccttccaagaaaccccgatcgtggaggtgagcagatccatcacgaagcacaactacctcatcctcgacgtcgacgacatcccccgcgtcgtcgccgaggctttcttcgtcgccacctccggccgccccggtccggtcctcatcgacattcccaaagacgttcagcagcaactcgccgtgcctaattgggacgagcccgttaacctccccggttacctcgccaggctgcccaggccccccgccgaggcccaattggaacacattgtcagactcatcatggaggcccaaaagcccgttctctacgtcggcggtggcagtttgaattccagtgctgaattgaggcgctttgttgaactcactggtattcccgttgctagcactttaatgggtcttggaacttttcctattggtgatgaatattcccttcagatgctgggtatgcatggtactgtttatgctaactatgctgttgacaatagtgatttgttgcttgcctttggggtaaggtttgatgaccgtgttactgggaagcttgaggcttttgctagtagggctaagattgttcacattgatattgattctgccgagattgggaagaacaagcaggcgcacgtgtcggtttgcgcggatttgaagttggccttgaagggaattaatatgattttggaggagaaaggagtggagggtaagtttgatcttggaggttggagagaagagattaatgtgcagaaacacaagtttccattgggttacaagacattccaggacgcgatttctccgcagcatgctatcgaggttcttgatgagttgactaatggagatgctattgttagtactggggttgggcagcatcaaatgtgggctgcgcagttttacaagtacaagagaccgaggcagtggttgacctcagggggtcttggagccatgggttttggattgcctgcggctattggtgctgctgttgctaaccctggggctgttgtggttgacattgatggggatggtagtttcatcatgaatgttcaggagttggccactataagagtggagaatctcccagttaagatattgttgttgaacaatcagcatttgggtatggtggttcagttggaggataggttctacaagtccaatagagctcacacctatcttggagatccgtctagcgagagcgagatattcccaaacatgctcaagtttgctgatgcttgtgggataccggcagcgcgagtgacgaagaaggaagagcttagagcggcaattcagagaatgttggacacccctggcccctaccttcttgatgtcattgtgccccatcaggagcatgtgttgccgatgattcccagtaatggatccttcaaggatgtgataactgagggtgatggtagaacgaggtactgattgcctagaccaaatgttccttgatgcttgttttgtacaatatatataagataatgctgtcctagttgcaggatttggcctgtggtgagcatcatagtctgtagtagttttggtagcaagacattttattttccttttatttaacttactacatgcagtagcatctatctatctctgtagtctgatatctcctgttgtctgtattgtgccgttggattttttgctgtagtgagactgaaaatgatgtgctagtaataatatttctgttagaaatctaagtagagaatctgttgaagaagtcaaaagctaatggaatcaggttacatattcaatgtttttctttttttagcggttggtagacgtgtagattcaacttctcttggagctcacctaggcaatcagtaaaatgcatattccttttttaacttgccatttatttacttttagtggaaattgtgaccaatttgttcatgtagaacggatttggaccattgcgtccacaaaacgtctcttttgctcgatcttcacaaagcgataccgaaatccagagatagttttcaaaagtcagaaatggcaaagttataaatagtaaaacagaatagatgctgtaatcgacttcaataacaagtggcatcacgtttctagttctagacccatcagctgggccggcccagctgatgatcccggtgaagttcctattccgaagttcctattctccagaaagtataggaacttcactagagcttgcggccgcgcatgctgacttaatcagctaacgccactcgagggggggcccggtaccggcgcgccgttctatagtgtcacctaaatcgtatgtgtatgatacataaggttatgtattaattgtagccgcgttctaacgacaatatgtccatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcaggttgatcagatctcgatcccgcgaaattaatacgactcactatagggagaccacaacggtttccctctagaaataattttgtttaactttaagaaggagatatacccatggaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggctatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagtgaggtacagcttggatcgatccggctgctaacaaagcccgaaaggaagctgagttggctgctgccaccgctgagcaataactagcataaccccttggggcctctaaacgggtcttgaggggttttttgctgaaaggaggaactatatccggatgctcgggcgcgccggtacccgggtaccgagctcactagacgcggtgaaattacctaattaacaccggtgtttatcgaaccactttgtacaagaaagctgggtctagatatctcgac

SEQ ID NO: 20 - DNA, 3933 bp, Artificial sequence
  QC489-1
ggtctggatcagacatcatatggatgctttcaaattcatgcgttggagattaattttactcataataggtaattatattaattaaaagaaattttacataaaaatacaacataaattattccattaaatatattattccctgtgactacaatgagataatctaagtgtatttgaaagtggaacagtagaaattataaaaattgcaatgagttgaataaaaaaggttggattaagaaagtaatctaagtacatttggaagtggaatagtagaaataaaattaaatgagttgaaattgaaaataattaaaaaaagtagggctaagaaatttctccttcaacttcatgatagcaaatattccattaggccatttgtagtttatgaatgagtatatataatcatgattttaggaattcgatctgctcgacacaaccgtgttacactttttttaaaatgtcatcataaaaataaaaaataaaagacatgttataattaagaataaggtgatcagtataaaaataagtaattttgggaaatattaaagttcaaaaaagaactattgaaagaaagaatattattatttaaaaagagaaaagaaaatgatgaaatgctattttcagttaaagaaaataagaaaaaaaaatacaaagaataattcaatgctggggctgtatatatgtttaagatgataattttttttttttttaaaaaaagataagaattaaatattttctcctttaatttctgaatcacggttttggttctgataagacactgattagtcacccatcaaatataatgaactaattctcctattctatttcaaaattttgattatacttagattaattttctaatatacttggacctgtttttcatgcagaagatgcagatatagctagacagcacctagtaatcgtggaaccaacaccaatgtccatatcatgcatgtgtgccacctttcaaatgtaatccagtagtaaaaaaagccatgacatgtaactccacgacagagtaaaactctcagaagtacctctcgtttcatatctgcaaatcctctaatataaataactcacttcacgggttcttttctcttcacagcaaaaacaattaataacaagggcgaattcgacccagctttcttgtacaaagttggcattataaaaaataattgctcatcaatttgttgcaacgaacaggtcactatcagtcaaaataaaatcattatttgccatccagctgatatcccctatagtgagtcgtattacatggtcatagctgtttcctggcagctctggcccgtgtctcaaaatctctgatgttacattgcacaagataaaaatatatcatcatgcctcctctagaccagccaggacagaaatgcctcgacttcgctgctgcccaaggttgccgggtgacgcacaccgtggaaacggatgaaggcacgaacccagtggacataagcctgttcggttcgtaagctgtaatgcaagtagcgtatgcgctcacgcaactggtccagaaccttgaccgaacgcagcggtggtaacggcgcagtggcggttttcatggcttgttatgactgtttttttggggtacagtctatgcctcgggcatccaagcagcaagcgcgttacgccgtgggtcgatgtttgatgttatggagcagcaacgatgttacgcagcagggcagtcgccctaaaacaaagttaaacatcatgagggaagcggtgatcgccgaagtatcgactcaactatcagaggtagttggcgtcatcgagcgccatctcgaaccgacgttgctggccgtacatttgtacggctccgcagtggatggcggcctgaagccacacagtgatattgatttgctggttacggtgaccgtaaggcttgatgaaacaacgcggcgagctttgatcaacgaccttttggaaacttcggcttcccctggagagagcgagattctccgcgctgtagaagtcaccattgttgtgcacgacgacatcattccgtggcgttatccagctaagcgcgaactgcaatttggagaatggcagcgcaatgacattcttgcaggtatcttcgagccagccacgatcgacattgatctggctatcttgctgacaaaagcaagagaacatagcgttgccttggtaggtccagcggcggaggaactctttgatccggttcctgaacaggatctatttgaggcgctaaatgaaaccttaacgctatggaactcgccgcccgactgggctggcgatgagcgaaatgtagtgcttacgttgtcccgcatttggtacagcgcagtaaccggcaaaatcgcgccgaaggatgtcgctgccgactgggcaatggagcgcctgccggcccagtatcagcccgtcatacttgaagctagacaggcttatcttggacaagaagaagatcgcttggcctcgcgcgcagatcagttggaagaatttgtccactacgtgaaaggcgagatcaccaaggtagtcggcaaataaccctcgagccacccatgaccaaaatcccttaacgtgagttacgcgtcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatacgcgtaccgctagccaggaagagtttgtagaaacgcaaaaaggccatccgtcaggatggccttctgcttagtttgatgcctggcagtttatggcgggcgtcctgcccgccaccctccgggccgttgcttcacaacgttcaaatccgctcccggcggatttgtcctactcaggagagcgttcaccgacaaacaacagataaaacgaaaggcccagtcttccgactgagcctttcgttttatttgatgcctggcagttccctactctcgcgttaacgctagcatggatgttttcccagtcacgacgttgtaaaacgacggccagtcttaagctcgggccccaaataatgattttattttgactgatagtgacctgttcgttgcaacaaattgatgagcaatgcttttttataatgccaactttgtacaaaaaagcaggctccgaattcgccctt

SEQ ID NO: 21 - DNA, 5286 bp, Artificial sequence
  QC330
atcaacaagtttgtacaaaaaagctgaacgagaaacgtaaaatgatataaatatcaatatattaaattagattttgcataaaaaacagactacataatactgtaaaacacaacatatccagtcatattggcggccgcattaggcaccccaggctttacactttatgcttccggctcgtataatgtgtggattttgagttaggatccgtcgagattttcaggagctaaggaagctaaaatggagaaaaaaatcactggatataccaccgttgatatatcccaatggcatcgtaaagaacattttgaggcatttcagtcagttgctcaatgtacctataaccagaccgttcagctggatattacggcctttttaaagaccgtaaagaaaaataagcacaagttttatccggcctttattcacattcttgcccgcctgatgaatgctcatccggaattccgtatggcaatgaaagacggtgagctggtgatatgggatagtgttcacccttgttacaccgttttccatgagcaaactgaaacgttttcatcgctctggagtgaataccacgacgatttccggcagtttctacacatatattcgcaagatgtggcgtgttacggtgaaaacctggcctatttccctaaagggtttattgagaatatgtttttcgtctcagccaatccctgggtgagtttcaccagttttgatttaaacgtggccaatatggacaacttcttcgcccccgttttcaccatgggcaaatattatacgcaaggcgacaaggtgctgatgccgctggcgattcaggttcatcatgccgtttgtgatggcttccatgtcggcagaatgcttaatgaattacaacagtactgcgatgagtggcagggcggggcgtaaagatctggatccggcttactaaaagccagataacagtatgcgtatttgcgcgctgatttttgcggtataagaatatatactgatatgtatacccgaagtatgtcaaaaagaggtatgctatgaagcagcgtattacagtgacagttgacagcgacagctatcagttgctcaaggcatatatgatgtcaatatctccggtctggtaagcacaaccatgcagaatgaagcccgtcgtctgcgtgccgaacgctggaaagcggaaaatcaggaagggatggctgaggtcgcccggtttattgaaatgaacggctcttttgctgacgagaacaggggctggtgaaatgcagtttaaggtttacacctataaaagagagagccgttatcgtctgtttgtggatgtacagagtgatattattgacacgcccgggcgacggatggtgatccccctggccagtgcacgtctgctgtcagataaagtctcccgtgaactttacccggtggtgcatatcggggatgaaagctggcgcatgatgaccaccgatatggccagtgtgccggtctccgttatcggggaagaagtggctgatctcagccaccgcgaaaatgacatcaaaaacgccattaacctgatgttctggggaatataaatgtcaggctcccttatacacagccagtctgcaggtcgaccatagtgactggatatgttgtgttttacagtattatgtagtctgttttttatgcaaaatctaatttaatatattgatatttatatcattttacgtttctcgttcagctttcttgtacaaagtggttgatgggatccatggcccacagcaagcacggcctgaaggaggagatgaccatgaagtaccacatggagggctgcgtgaacggccacaagttcgtgatcaccggcgagggcatcggctaccccttcaagggcaagcagaccatcaacctgtgcgtgatcgagggcggccccctgcccttcagcgaggacatcctgagcgccggcttcaagtacggcgaccggatcttcaccgagtacccccaggacatcgtggactacttcaagaacagctgccccgccggctacacctggggccggagcttcctgttcgaggacggcgccgtgtgcatctgtaacgtggacatcaccgtgagcgtgaaggagaactgcatctaccacaagagcatcttcaacggcgtgaacttccccgccgacggccccgtgatgaagaagatgaccaccaactgggaggccagctgcgagaagatcatgcccgtgcctaagcagggcatcctgaagggcgacgtgagcatgtacctgctgctgaaggacggcggccggtaccggtgccagttcgacaccgtgtacaaggccaagagcgtgcccagcaagatgcccgagtggcacttcatccagcacaagctgctgcgggaggaccggagcgacgccaagaaccagaagtggcagctgaccgagcacgccatcgccttccccagcgccctggcctgagagctcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattctagtggccggcccagctgatatccatcacactggcggccgctcgagttctatagtgtcacctaaatcgtatgtgtatgatacataaggttatgtattaattgtagccgcgttctaacgacaatatgtccatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcaggttgatcagatctcgatcccgcgaaattaatacgactcactatagggagaccacaacggtttccctctagaaataattttgtttaactttaagaaggagatatacccatggaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggctatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagtgaggtacagcttggatcgatccggctgctaacaaagcccgaaaggaagctgagttggctgctgccaccgctgagcaataactagcataaccccttggggcctctaaacgggtcttgaggggttttttgctgaaaggaggaactatatccggatgatcgtcgaggcctcacgtgttaacaagcttgcatgcctgcaggttt

SEQ ID NO: 22 - DNA, 4774 bp, Artificial sequence
  QC489-1Y
ggtctggatcagacatcatatggatgctttcaaattcatgcgttggagattaattttactcataataggtaattatattaattaaaagaaattttacataaaaatacaacataaattattccattaaatatattattccctgtgactacaatgagataatctaagtgtatttgaaagtggaacagtagaaattataaaaattgcaatgagttgaataaaaaaggttggattaagaaagtaatctaagtacatttggaagtggaatagtagaaataaaattaaatgagttgaaattgaaaataattaaaaaaagtagggctaagaaatttctccttcaacttcatgatagcaaatattccattaggccatttgtagtttatgaatgagtatatataatcatgattttaggaattcgatctgctcgacacaaccgtgttacactttttttaaaatgtcatcataaaaataaaaaataaaagacatgttataattaagaataaggtgatcagtataaaaataagtaattttgggaaatattaaagttcaaaaaagaactattgaaagaaagaatattattatttaaaaagagaaaagaaaatgatgaaatgctattttcagttaaagaaaataagaaaaaaaaatacaaagaataattcaatgctggggctgtatatatgtttaagatgataattttttttttttttaaaaaaagataagaattaaatattttctcctttaatttctgaatcacggttttggttctgataagacactgattagtcacccatcaaatataatgaactaattctcctattctatttcaaaattttgattatacttagattaattttctaatatacttggacctgtttttcatgcagaagatgcagatatagctagacagcacctagtaatcgtggaaccaacaccaatgtccatatcatgcatgtgtgccacctttcaaatgtaatccagtagtaaaaaaagccatgacatgtaactccacgacagagtaaaactctcagaagtacctctcgtttcatatctgcaaatcctctaatataaataactcacttcacgggttcttttctcttcacagcaaaaacaattaataacaagggcgaattcgacccagctttcttgtacaaagtggttgatgggatccatggcccacagcaagcacggcctgaaggaggagatgaccatgaagtaccacatggagggctgcgtgaacggccacaagttcgtgatcaccggcgagggcatcggctaccccttcaagggcaagcagaccatcaacctgtgcgtgatcgagggcggccccctgcccttcagcgaggacatcctgagcgccggcttcaagtacggcgaccggatcttcaccgagtacccccaggacatcgtggactacttcaagaacagctgccccgccggctacacctggggccggagcttcctgttcgaggacggcgccgtgtgcatctgtaacgtggacatcaccgtgagcgtgaaggagaactgcatctaccacaagagcatcttcaacggcgtgaacttccccgccgacggccccgtgatgaagaagatgaccaccaactgggaggccagctgcgagaagatcatgcccgtgcctaagcagggcatcctgaagggcgacgtgagcatgtacctgctgctgaaggacggcggccggtaccggtgccagttcgacaccgtgtacaaggccaagagcgtgcccagcaagatgcccgagtggcacttcatccagcacaagctgctgcgggaggaccggagcgacgccaagaaccagaagtggcagctgaccgagcacgccatcgccttccccagcgccctggcctgagagctcgaatttccccgatcgttcaaacatttggcaataaagtttcttaagattgaatcctgttgccggtcttgcgatgattatcatataatttctgttgaattacgttaagcatgtaataattaacatgtaatgcatgacgttatttatgagatgggtttttatgattagagtcccgcaattatacatttaatacgcgatagaaaacaaaatatagcgcgcaaactaggataaattatcgcgcgcggtgtcatctatgttactagatcgggaattctagtggccggcccagctgatatccatcacactggcggccgctcgagttctatagtgtcacctaaatcgtatgtgtatgatacataaggttatgtattaattgtagccgcgttctaacgacaatatgtccatatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgtccttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagcattgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcaggttgatcagatctcgatcccgcgaaattaatacgactcactatagggagaccacaacggtttccctctagaaataattttgtttaactttaagaaggagatatacccatggaaaagcctgaactcaccgcgacgtctgtcgagaagtttctgatcgaaaagttcgacagcgtctccgacctgatgcagctctcggagggcgaagaatctcgtgctttcagcttcgatgtaggagggcgtggatatgtcctgcgggtaaatagctgcgccgatggtttctacaaagatcgttatgtttatcggcactttgcatcggccgcgctcccgattccggaagtgcttgacattggggaattcagcgagagcctgacctattgcatctcccgccgtgcacagggtgtcacgttgcaagacctgcctgaaaccgaactgcccgctgttctgcagccggtcgcggaggctatggatgcgatcgctgcggccgatcttagccagacgagcgggttcggcccattcggaccgcaaggaatcggtcaatacactacatggcgtgatttcatatgcgcgattgctgatccccatgtgtatcactggcaaactgtgatggacgacaccgtcagtgcgtccgtcgcgcaggctctcgatgagctgatgctttgggccgaggactgccccgaagtccggcacctcgtgcacgcggatttcggctccaacaatgtcctgacggacaatggccgcataacagcggtcattgactggagcgaggcgatgttcggggattcccaatacgaggtcgccaacatcttcttctggaggccgtggttggcttgtatggagcagcagacgcgctacttcgagcggaggcatccggagcttgcaggatcgccgcggctccgggcgtatatgctccgcattggtcttgaccaactctatcagagcttggttgacggcaatttcgatgatgcagcttgggcgcagggtcgatgcgacgcaatcgtccgatccggagccgggactgtcgggcgtacacaaatcgcccgcagaagcgcggccgtctggaccgatggctgtgtagaagtactcgccgatagtggaaaccgacgccccagcactcgtccgagggcaaaggaatagtgaggtacagcttggatcgatccggctgctaacaaagcccgaaaggaagctgagttggctgctgccaccgctgagcaataactagcataaccccttggggcctctaaacgggtcttgaggggttttttgctgaaaggaggaactatatccggatgatcgtcgaggcctcacgtgttaacaagcttgcatgcctgcaggtttatcaacaagtttgtacaaaaaagcaggctccgaattcgccctt

SEQ ID NO: 23 - DNA, 26 bp, Artificial sequence
  Sams-L primer
gaccaagacacactcgttcatatatc

SEQ ID NO: 24 - DNA, 25 bp, Artificial sequence
  Sams-L2 primer
tctgctgctcaatgtttacaaggac

SEQ ID NO: 25 - DNA, 27 bp, Artificial sequence
  PSO333255 sense primer
ccactcagattgtaagagctgtatgtg

SEQ ID NO: 26 - DNA, 22 bp, Artificial sequence
  SO333255 antisense primer
gcagaagtcgttggtgtcaaga

SEQ ID NO: 27 - DNA, 24 bp, Artificial sequence
  ATPS sense primer
catgattgggagaaaccttaagct

SEQ ID NO: 28 - DNA, 20 bp, Artificial sequence
  ATPS antisense primer
agattgggccagaggatcct

SEQ ID NO: 29 - DNA, 22 bp, Artificial sequence
  SAMS forward primer (SAMS-48F)
ggaagaagagaatcgggtggtt

SEQ ID NO: 30 - DNA, 23 bp, Artificial sequence
  FAM labeled SAMS probe(SAMS-88T)
attgtgttgtgtggcatggttat

SEQ ID NO: 31 - DNA, 23 bp, Artificial sequence
  SAMS reverse prime (SAMS-134R
ggcttgttgtgcagtttttgaag

SEQ ID NO: 32 - DNA, 20 bp, Artificial sequence
  YFP forward primer(YFP-67F
aacggccacaagttcgtgat

SEQ ID NO: 33 - DNA, 20 bp, Artificial sequence
  FAM labeled YFP probe(YFP-88T)
accggcgagggcatcggcta

SEQ ID NO: 34 - DNA, 20 bp, Artificial sequence
  YFP reverse primer(YFP-130R)
cttcaagggcaagcagacca

SEQ ID NO: 35 - DNA, 24 bp, Artificial sequence
  HSP forward primer(HSP-F1)
caaacttgacaaagccacaactct

SEQ ID NO: 36 - DNA, 20 bp, Artificial sequence
  VIC labeled HSP probe(HSP probe)
ctctcatctcatataaatac

SEQ ID NO: 37 - DNA, 21 bp, Artificial sequence
  HSP reverse primer(HSP-R1)
ggagaaattggtgtcgtggaa

SEQ ID NO: 38 - DNA, 100 bp, Artificial sequence
  AttL1
caaataatgattttattttgactgatagtgacctgttcgttgcaacaaattgataagcaatgcttttttataatgccaactttgtacaaaaaagcaggct

SEQ ID NO: 39 - DNA, 100 bp, Artificial sequence
  AttL2;
caaataatgattttattttgactgatagtgacctgttcgttgcaacaaattgataagcaatgctttcttataatgccaactttgtacaagaaagctgggt

SEQ ID NO: 40 - DNA, 125 bp, Artificial sequence
  AttR1
acaagtttgtacaaaaaagctgaacgagaaacgtaaaatgatataaatatcaatatattaaattagattttgcataaaaaacagactacataatactgtaaaacacaacatatccagtcactatg

SEQ ID NO: 41 - DNA, 125 bp, Artificial sequence
  AttR2
accactttgtacaagaaagctgaacgagaaacgtaaaatgatataaatatcaatatattaaattagattttgcataaaaaacagactacataatactgtaaaacacaacatatccagtcactatg

SEQ ID NO: 42 - DNA, 21 bp, Artificial sequence
  AttB1
caagtttgtacaaaaaagcag

SEQ ID NO: 43 - DNA, 21 bp, Artificial sequence
  AttB2
cagctttcttgtacaaagtgg

SEQ ID NO: 44 - DNA, 487 bp, Glycine max
cttcacagcaaaaacaattaataaagatgagtttgaagaacaacatggtggtgctaaaggtgtgtttgttgcttcttttccttgtgggggttacagctgcacgcatggaactgagcttcttcaaaagtgatcagtcatcaagttatgatgatgatgagtattcaaaaccatgctgtgatctctgcatgtgcacacgctcaatgcctcctcaatgcagctgtgaagatattaggctgaattcatgccactcagattgtaagagctgtatgtgcacacgctcacagccaggacagtgtcgttgtcttgacaccaacgacttctgctacaaaccttgcaagtccagagatgactagaaaaactaatagctctctcaaatggacgaagcccctttaggctttgtttgttatgttaggggagacaaataaaacaagaaataaaagctcagtggccagtaatttgcttttagcaaatttggtcatttttacag

SEQ ID NO: 45 - protein, 108 aa, Glycine max; identical to SEQ ID NO: 16

(19) United States
(12) Patent Application Publication
(10) Pub. No.: US 2014/0366218 A1
(43) Pub. Date: Dec. 11, 2014

(54) SOYBEAN BB13 PROMOTER AND ITS USE IN EMBRYO-SPECIFIC EXPRESSION OF TRANSGENIC GENES IN PLANTS

(71) Applicant: Zhongsen Li, Hockessin, DE (US)

(72) Inventor: Zhongsen Li, Hockessin, DE (US)

(73) Assignee: E I DU PONT DE NEMOURS AND COMPANY, Wilmington, DE (US)

(21) Appl. No.: 14/344,333

(22) PCT Filed: Sep. 13, 2012
(86) PCT No.: PCT/US12/55236

§ 371 Date: Mar. 12, 2014

Related U.S. Application Data
(60) Provisional application No. 61/533,826, filed on Sep. 13, 2011.

(51) Int. Cl. C12N 15/82

(52) U.S. Cl. CPC C12N 15/8234; C12N 15/8279; C12N 15/8274; C12N 15/8286; C12N 15/8245; C12N 15/8247; C12N 15/8251; C12N 15/8261; C12N 15/8273; C12N 15/8271
USPC 800/279; 435/320.1; 435/419; 800/298; 800/312; 800/278; 435/468; 800/284; 800/281; 800/290; 800/289

(57) ABSTRACT

The invention relates to gene expression regulatory sequences from soybean, specifically to the promoter of a soybean Bowman-Birk type proteinase isoinhibitor gene (BBI3) and fragments thereof and their use in promoting the expression of one or more heterologous nucleotide sequences in an embryo-specific manner in plants. The invention further discloses compositions, polynucleotide constructs, transformed host cells, transgenic plants and seeds containing the recombinant construct with the promoter, and methods for preparing and using the same.

This application claims the benefit of U.S. Patent Application Ser. No. 61/533,826, filed Sep. 13, 2011, which is herein incorporated by reference in their entirety.

### FIELD OF THE INVENTION

This invention relates to a plant promoter GM-BBI3 and fragments thereof and their use in altering expression of at least one heterologous nucleic acid fragment in plants.

### BACKGROUND OF THE INVENTION

Recent advances in plant genetic engineering have opened new doors to engineer plants to have improved characteristics or traits, such as plant disease resistance, insect resistance, herbicidal resistance, yield improvement, improvement of the nutritional quality of the edible portions of the plant, and enhanced stability or shelf-life of the ultimate consumer product obtained from the plants. Thus, a desired gene (or genes) with the molecular function to impart different or improved characteristics or qualities can be incorporated properly into the plant's genome. The newly integrated gene (or genes) coding sequence can then be expressed in the plant cell to exhibit the desired new trait or characteristic. It is important that appropriate regulatory signals be present in proper configurations in order to obtain the expression of the newly inserted gene coding sequence in the plant cell. These regulatory signals typically include a promoter region, a 5′ non-translated leader sequence and a 3′ transcription termination/polyadenylation sequence.

A promoter is a non-coding genomic DNA sequence, usually upstream (5′) to the relevant coding sequence, to which RNA polymerase binds before initiating transcription. This binding aligns the RNA polymerase so that transcription will initiate at a specific transcription initiation site. The nucleotide sequence of the promoter determines the nature of the RNA polymerase binding and other related protein factors that attach to the RNA polymerase and/or promoter, and the rate of RNA synthesis.

It has been shown that certain promoters are able to direct RNA synthesis at a higher rate than others. These are called “strong promoters”. Certain other promoters have been shown to direct RNA synthesis at higher levels only in particular types of cells or tissues and are often referred to as “tissue specific promoters”, or “tissue-preferred promoters”, if the promoters direct RNA synthesis preferentially in certain tissues (RNA synthesis may occur in other tissues at reduced levels). Since patterns of expression of a chimeric gene (or genes) introduced into a plant are controlled using promoters, there is an ongoing interest in the isolation of novel promoters that are capable of controlling the expression of a chimeric gene (or genes) at certain levels in specific tissue types or at specific plant developmental stages.

Although advances in technology provide greater success in transforming plants with chimeric genes, there is still a need for specific expression of such genes in desired plants. Often times it is desired to selectively express target genes in a specific tissue because of toxicity or efficacy concerns. For example, embryo tissue is a type of tissue where specific expression is desirable and there remains a need for promoters that preferably initiate transcription in embryo tissue. Promoters that initiate transcription preferably in embryo tissue control genes involved in embryo and seed development.

### SUMMARY OF THE INVENTION

This invention concerns an isolated polynucleotide comprising a promoter region of the Bowman-Birk type proteinase isoinhibitor D protein (BBI3) Glycine max gene as set forth in SEQ ID NO:1, wherein said promoter comprises a deletion at the 5′-terminus of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, 1000, 1001, 1002, 1003, 1004, 1005, 1006, 1007, 1008, 1009, 1010, 1011, 1012, 1013, 1014, 1015, 1016, 1017, 1018, 1019, 1020, 1021, 1022, 1023, 1024, 1025, 1026, 1027, 1028, 1029, 1030, 1031, 1032, 1033, 1034, 1035, 1036, 1037, 1038, 1039, 1040, 1041, 1042, 1043, 1044, 1045, 1046, 1047, 1048, 1049, 1050, 1051, 1052, 1053, 1054, 1055, 1056, 1057, 1058, 1059, 1060, 1061, 1062, 1063, 1064, 1065, 1066, 1067, 1068, 1069, 1070, 1071, 1072, 1073, 1074, 1075, 1076, 1077, 1078, 1079, 1080, 1081, 1082, 1083, 1084, 1085, 1086, 1087, 1088, 1089, 1090, 1091, 1092, 1093, 1094, 1095, 1096, 1097, 1098, 1099, 1100, 1101, 1102, 1103, 1104, 1105, 1106, 1107, 1108, 1109, 1110, 1111, 1112, 1113, 1114, 1115, 1116, 1117 or 1118 consecutive nucleotides, wherein the first nucleotide deleted is the cytosine nucleotide [‘C’ ] at position 1 of SEQ ID NO:1. This invention also concerns the isolated polynucleotide of claim 1, wherein the polynucleotide is an embryo specific promoter.

In a second embodiment, this invention concerns an isolated polynucleotide comprising a promoter wherein said promoter comprises the nucleotide sequence set forth in SEQ ID NOs: 1, 2, 3, 4, 5, or 6 or said promoter consists essentially of a fragment that is substantially similar and functionally equivalent to the nucleotide sequence set forth in SEQ ID NOs: 1, 2, 3, 4, 5, or 6.

In a third embodiment, this invention concerns a recombinant expression construct comprising at least one heterologous nucleotide sequence operably linked to the promoter of the invention.

In a fourth embodiment, this invention concerns a cell, plant, or seed comprising a recombinant DNA construct of the present disclosure.

In a fifth embodiment, this invention concerns plants comprising this recombinant DNA construct and seeds obtained from such plants.

In a sixth embodiment, this invention concerns a method of altering (increasing or decreasing) expression of at least one heterologous nucleic acid fragment in a plant cell which comprises:
(a) transforming a plant cell with the recombinant expression construct described above;
(b) growing fertile mature plants from the transformed plant cell of step (a);
(c) selecting plants containing the transformed plant cell wherein the expression of the heterologous nucleic acid fragment is increased or decreased.

In a seventh embodiment, this invention concerns a method for expressing a yellow fluorescent protein ZS-YELLOW1 N1 in a host cell comprising:
(a) transforming a host cell with a recombinant expression construct comprising at least one ZS-YELLOW1 N1 (YFP) nucleic acid fragment operably linked to a promoter wherein said promoter consists essentially of the nucleotide sequence set forth in SEQ ID NOs:1, 2, 3, 4, 5, or 6; and
(b) growing the transformed host cell under conditions that are suitable for expression of the recombinant DNA construct, wherein expression of the recombinant DNA construct results in production of increased levels of ZS-YELLOW1 N1 protein in the transformed host cell when compared to a corresponding nontransformed host cell.

In an eighth embodiment, this invention concerns an isolated nucleic acid fragment comprising a plant Bowman-Birk type proteinase isoinhibitor D protein (BBI3) gene promoter.

In a ninth embodiment, this invention concerns a method of altering a marketable plant trait. The marketable plant trait concerns genes and proteins involved in disease resistance, herbicide resistance, insect resistance, carbohydrate metabolism, fatty acid metabolism, amino acid metabolism, plant development, plant growth regulation, yield improvement, drought resistance, cold resistance, heat resistance, and salt resistance.

In a tenth embodiment, this invention concerns an isolated polynucleotide linked to a heterologous nucleic acid sequence. The heterologous nucleic acid sequence encodes a protein involved in disease resistance, herbicide resistance, insect resistance; carbohydrate metabolism, fatty acid metabolism, amino acid metabolism, plant development, plant growth regulation, yield improvement, drought resistance, cold resistance, heat resistance, or salt resistance in plants.

### BRIEF DESCRIPTION OF THE DRAWINGS AND SEQUENCE LISTINGS

The invention can be more fully understood from the following detailed description and the accompanying drawings and Sequence Listing that form a part of this application.

FIG. 1A is the logarithm of relative quantifications of the soybean Bowman-Birk type proteinase isoinhibitor D protein gene (PSO333255) expression in 14 soybean tissues by quantitative RT-PCR. The gene expression profile indicates that the BBI3 gene is highly expressed in developed seeds and somatic embryos but not significantly in other checked tissues.

FIG. 1B is the relative expression of the soybean Bowman-Birk type proteinase isoinhibitor D protein gene (Glyma16g33400.1) gene in twenty soybean tissues by Illumina (Solexa) digital gene expression dual-tag-based mRNA profiling. The gene expression profile indicates that the BBI3 gene is highly expressed in developed full size and mature seeds and in mature somatic embryos but not in other checked tissues.

FIG. 2 is BBI3 promoter copy number analysis by Southern.

FIG. 3A-3B shows the maps of plasmid pCR2.1-TOPO, QC489, QC478i, and QC607.

FIG. 4A-4B shows the maps of plasmid pCR8/GW/TOPO, QC489-1, QC300, and QC489-1Y containing the truncated 1116 bp BBI3 promoter. Promoter deletion constructs QC489-2Y, QC489-3Y, QC489-4Y, and QC489-5Y containing the 885, 698, 473, and 245 bp truncated BBI3 promoters, respectively, have the same map configuration, except for the truncated promoter sequences.

FIG. 5 is the schematic description of the full length construct QC489 and its progressive truncation constructs, QC489-1Y, QC489-2Y, QC489-3Y, QC489-4Y, and QC489-5Y, of the BBI3 promoter. The size of each promoter is given at the left end of each drawing.

FIG. 6 is the transient expression of the fluorescent protein reporter gene ZS-YELLOW1 N1 in the cotyledons of germinating soybean seeds (shown as white spots). The reporter gene is driven by the full length BBI3 promoter in QC489 or by progressively truncated BBI3 promoters in the transient expression constructs QC489-1Y to QC489-5Y.

FIG. 7A-7P shows the stable expression of the fluorescent protein reporter gene ZS-YELLOW1 N1 in transgenic soybean plants containing a single copy of the transgene construct QC607. White areas (yellow in color display) indicate gene ZS-YELLOW1 N1 gene expression. Gray (red in color display) is background auto fluorescence from plant green tissues. FIG. 7 P shows highest fluorescence in the embryo tissue further supporting that this promoter is an embryo-specific promoter.

The sequence descriptions summarize the Sequence Listing attached hereto. The Sequence Listing contains one letter codes for nucleotide sequence characters and the single and three letter codes for amino acids as defined in the IUPAC-IUB standards described in Nucleic Acids Research 13:3021-3030 (1985) and in the Biochemical Journal 219(2):345-373 (1984).

SEQ ID NO:1 is the DNA sequence comprising a 1363 bp (base pair) soybean BBI3 promoter.

SEQ ID NO:2 is a 1116 bp truncated form of the BBI3 promoter shown in SEQ ID NO:1 (bp 241-1357 of SEQ ID NO:1).

SEQ ID NO:3 is a 885 bp truncated form of the BBI3 promoter shown in SEQ ID NO:1 (bp 472-1357 of SEQ ID NO:1).

SEQ ID NO:4 is a 698 bp truncated form of the BBI3 promoter shown in SEQ ID NO:1 (bp 659-1357 of SEQ ID NO:1).

SEQ ID NO:5 is a 473 bp truncated form of the BBI3 promoter shown in SEQ ID NO:1 (bp 884-1357 of SEQ ID NO:1).

SEQ ID NO:6 is a 245 bp truncated form of the BBI3 promoter shown in SEQ ID NO:1 (bp 1112-1357 of SEQ ID NO:1).

SEQ ID NO:7 is an oligonucleotide primer used as a sense primer in the PCR amplification of the full length BBI3 promoter in SEQ ID NO:1 when paired with SEQ ID NO:8. A restriction enzyme XmaI recognition site CCCGGG is included for subsequent cloning.

SEQ ID NO:8 is an oligonucleotide primer used as an antisense primer in the PCR amplification of the full length BBI3 promoter in SEQ ID NO:1 when paired with SEQ ID NO:7. A restriction enzyme NcoI recognition site CCATGG is included for subsequent cloning.

SEQ ID NO:9 is an oligonucleotide primer used as an antisense primer in the PCR amplifications of the truncated BBI3 promoters in SEQ ID NOs:2, 3, 4, 5, or 6 when paired with SEQ ID NOs:10, 11, 12, 13, or 14, respectively.

SEQ ID NO:10 is an oligonucleotide primer used as a sense primer in the PCR amplification of the truncated BBI3 promoter in SEQ ID NO:2 when paired with SEQ ID NO:9.

SEQ ID NO:11 is an oligonucleotide primer used as a sense primer in the PCR amplification of the truncated BBI3 promoter in SEQ ID NO:3 when paired with SEQ ID NO:9.

SEQ ID NO:12 is an oligonucleotide primer used as a sense primer in the PCR amplification of the truncated BBI3 promoter in SEQ ID NO:4 when paired with SEQ ID NO:9.

SEQ ID NO:13 is an oligonucleotide primer used as a sense primer in the PCR amplification of the truncated BBI3 promoter in SEQ ID NO:5 when paired with SEQ ID NO:9.

SEQ ID NO:14 is an oligonucleotide primer used as a sense primer in the PCR amplification of the truncated BBI3 promoter in SEQ ID NO:6 when paired with SEQ ID NO:9.

SEQ ID NO:15 is the 482 bp nucleotide sequence of the putative soybean Bowman-Birk type proteinase isoinhibitor D protein gene BBI3 (PSO0333255). Nucleotides 1 to 22 are the 5′ untranslated sequence, nucleotides 23 to 25 are the translation initiation codon, nucleotides 23 to 346 are the polypeptide coding region, nucleotides 347 to 349 are the termination codon, and nucleotides 350 to 482 are part of the 3′ untranslated sequence.

SEQ ID NO:16 is the predicted 108 aa (amino acid) long peptide sequence translated from the coding region of the soybean Bowman-Birk type proteinase isoinhibitor D protein gene BBI3 nucleotide sequence SEQ ID NO:15.

SEQ ID NO:17 is the 4640 bp sequence of QC489.

SEQ ID NO:18 is the 8482 bp sequence of QC478i.

SEQ ID NO:19 is the 9239 bp sequence of QC607.

SEQ ID NO:20 is the 3933 bp sequence of QC489-1.

SEQ ID NO:21 is the 5286 bp sequence of QC330.

SEQ ID NO:22 is the 4774 bp sequence of QC489-1Y.

SEQ ID NO:23 is an oligonucleotide primer used in the diagnostic PCR to check for soybean genomic DNA presence in total RNA or cDNA when paired with SEQ ID NO:24.

SEQ ID NO:24 is an oligonucleotide primer used in the diagnostic PCR to check for soybean genomic DNA presence in total RNA or cDNA when paired with SEQ ID NO:23.

SEQ ID NO:25 is a sense primer used in quantitative RT-PCR analysis of PSO333255 gene expression.

SEQ ID NO:26 is an antisense primer used in quantitative RT-PCR analysis of PSO333255 gene expression.

SEQ ID NO:27 is a sense primer used as an endogenous control gene primer in quantitative RT-PCR analysis of gene expression.

SEQ ID NO:28 is an antisense primer used as an endogenous control gene primer in quantitative RT-PCR analysis of gene expression.

SEQ ID NO:29 is a sense primer used in quantitative PCR analysis of SAMS:ALS transgene copy numbers.

SEQ ID NO:30 is a FAM labeled fluorescent DNA oligo probe used in quantitative PCR analysis of SAMS:ALS transgene copy numbers.

SEQ ID NO:31 is an antisense primer used in quantitative PCR analysis of SAMS:ALS transgene copy numbers.

SEQ ID NO:32 is a sense primer used in quantitative PCR analysis of GM-BBI3:YFP transgene copy numbers.

SEQ ID NO:33 is a FAM labeled fluorescent DNA oligo probe used in quantitative PCR analysis of GM-BBI3:YFP transgene copy numbers.

SEQ ID NO:34 is an antisense primer used in quantitative PCR analysis of GM-BBI3:YFP transgene copy numbers.

SEQ ID NO:35 is a sense primer used as an endogenous control gene primer in quantitative PCR analysis of transgene copy numbers.

SEQ ID NO:36 is a VIC labeled DNA oligo probe used as an endogenous control gene probe in quantitative PCR analysis of transgene copy numbers.

SEQ ID NO:37 is an antisense primer used as an endogenous control gene primer in quantitative PCR analysis of transgene copy numbers.

SEQ ID NO:38 is the recombination site attL1 sequence in the GATEWAY® cloning system (Invitrogen, Carlsbad, Calif.).

SEQ ID NO:39 is the recombination site attL2 sequence in the GATEWAY® cloning system (Invitrogen).

SEQ ID NO:40 is the recombination site attR1 sequence in the GATEWAY® cloning system (Invitrogen).

SEQ ID NO:41 is the recombination site attR2 sequence in the GATEWAY® cloning system (Invitrogen).

SEQ ID NO:42 is the recombination site attB1 sequence in the GATEWAY® cloning system (Invitrogen).

SEQ ID NO:43 is the recombination site attB2 sequence in the GATEWAY® cloning system (Invitrogen).

SEQ ID NO:44 is the nucleotide sequence of the Glycine max Bowman-Birk type proteinase isoinhibitor D protein gene (NCBI accession AB081836.1).

SEQ ID NO:45 is the amino acid sequence of the Glycine max Bowman-Birk type proteinase isoinhibitor D protein gene (NCBI accession BAB86786.1).

### DETAILED DESCRIPTION OF THE INVENTION

The disclosure of all patents, patent applications, and publications cited herein are incorporated by reference in their entirety.

As used herein and in the appended claims, the singular forms “a”, “an”, and “the” include plural reference unless the context clearly dictates otherwise. Thus, for example, reference to “a plant” includes a plurality of such plants, reference to “a cell” includes one or more cells and equivalents thereof known to those skilled in the art, and so forth.

In the context of this disclosure, a number of terms shall be utilized.

An “isolated polynucleotide” refers to a polymer of ribonucleotides (RNA) or deoxyribonucleotides (DNA) that is single- or double-stranded, optionally containing synthetic, non-natural or altered nucleotide bases. An isolated polynucleotide in the form of DNA may be comprised of one or more segments of cDNA, genomic DNA or synthetic DNA.

The terms “polynucleotide”, “polynucleotide sequence”, “nucleic acid sequence”, “nucleic acid fragment”, and “isolated nucleic acid fragment” are used interchangeably herein. These terms encompass nucleotide sequences and the like. A polynucleotide may be a polymer of RNA or DNA that is single- or double-stranded, that optionally contains synthetic, non-natural or altered nucleotide bases. A polynucleotide in the form of a polymer of DNA may be comprised of one or more segments of cDNA, genomic DNA, synthetic DNA, or mixtures thereof. Nucleotides (usually found in their 5′-monophosphate form) are referred to by a single letter designation as follows: “A” for adenylate or deoxyadenylate (for RNA or DNA, respectively), “C” for cytidylate or deoxycytidylate, “G” for guanylate or deoxyguanylate, “U” for uridylate, “T” for deoxythymidylate, “R” for purines (A or G), “Y” for pyrimidines (C or T), “K” for G or T, “H” for A or C or T, “I” for inosine, and “N” for any nucleotide.

As used herein, a “GM-BBI3 promoter” refers to the promoter of a putative Glycine max gene with significant homology to Bowman-Birk type proteinase isoinhibitor protein genes identified in various plant species including soybean that are deposited in National Center for Biotechnology Information (NCBI) databases (NCBI accession AB0081836.1; SEQ ID NO: 44).

“Promoter” refers to a nucleic acid fragment capable of controlling transcription of another nucleic acid fragment. A promoter is capable of controlling the expression of a coding sequence or functional RNA. Functional RNA includes, but is not limited to, transfer RNA (tRNA) and ribosomal RNA (rRNA). The promoter sequence consists of proximal and more distal upstream elements, the latter elements often referred to as enhancers. Accordingly, an “enhancer” is a DNA sequence that can stimulate promoter activity, and may be an innate element of the promoter or a heterologous element inserted to enhance the level or tissue-specificity of a promoter. Promoters may be derived in their entirety from a native gene, or be composed of different elements derived from different promoters found in nature, or even comprise synthetic DNA segments. It is understood by those skilled in the art that different promoters may direct the expression of a gene in different tissues or cell types, or at different stages of development, or in response to different environmental conditions. New promoters of various types useful in plant cells are constantly being discovered; numerous examples may be found in the compilation by Okamuro and Goldberg (Biochemistry of Plants 15:1-82 (1989)). It is further recognized that since in most cases the exact boundaries of regulatory sequences have not been completely defined, DNA fragments of some variation may have identical promoter activity.

“Promoter functional in a plant” is a promoter capable of controlling transcription in plant cells whether or not its origin is from a plant cell.

“Tissue-specific promoter” and “tissue-preferred promoter” are used interchangeably to refer to a promoter that is expressed predominantly but not necessarily exclusively in one tissue or organ, but that may also be expressed in one specific cell.

“Embryo-specific promoter” and “embryo-preferred promoter” are used interchangeably to refer to a promoter that is active during embryo development or expressed predominantly but not necessarily exclusively in embryo tissue.

“Developmentally regulated promoter” refers to a promoter whose activity is determined by developmental events.

“Constitutive promoter” refers to promoters active in all or most tissues or cell types of a plant at all or most developing stages. As with other promoters classified as “constitutive” (e.g. ubiquitin), some variation in absolute levels of expression can exist among different tissues or stages. The term “constitutive promoter” or “tissue-independent” are used interchangeably herein.

The promoter nucleotide sequences and methods disclosed herein are useful in regulating embryo-specific expression of any heterologous nucleotide sequences in a host plant in order to alter the phenotype of a plant.

A “heterologous nucleotide sequence” refers to a sequence that is not naturally occurring with the plant promoter sequence of the invention. While this nucleotide sequence is heterologous to the promoter sequence, it may be homologous, or native, or heterologous, or foreign, to the plant host. However, it is recognized that the instant promoters may be used with their native coding sequences to increase or decrease expression resulting in a change in phenotype in the transformed seed. The terms “heterologous nucleotide sequence”, “heterologous sequence”, “heterologous nucleic acid fragment”, and “heterologous nucleic acid sequence” are used interchangeably herein.

Among the most commonly used promoters are the nopaline synthase (NOS) promoter (Ebert et al., Proc. Natl. Acad. Sci. U.S.A. 84:5745-5749 (1987)), the octapine synthase (OCS) promoter, caulimovirus promoters such as the cauliflower mosaic virus (CaMV) 19S promoter (Lawton et al., Plant Mol. Biol. 9:315-324 (1987)), the CaMV 35S promoter (Odell et al., Nature 313:810-812 (1985)), and the figwort mosaic virus 35S promoter (Sanger et al., Plant Mol. Biol. 14:433-43 (1990)), the light inducible promoter from the small subunit of rubisco, the Adh promoter (Walker et al., Proc. Natl. Acad. Sci. U.S.A. 84:6624-66280 (1987), the sucrose synthase promoter (Yang et al., Proc. Natl. Acad. Sci. U.S.A. 87:4144-4148 (1990)), the R gene complex promoter (Chandler et al., Plant Cell 1:1175-1183 (1989)), the chlorophyll a/b binding protein gene promoter, etc. Other commonly used promoters are, the promoters for the potato tuber ADPGPP genes, the sucrose synthase promoter, the granule bound starch synthase promoter, the glutelin gene promoter, the maize waxy promoter, Brittle gene promoter, and Shrunken 2 promoter, the acid chitinase gene promoter, and the zein gene promoters (15 kD, 16 kD, 19 kD, 22 kD, and 27 kD; Perdersen et al., Cell 29:1015-1026 (1982)). A plethora of promoters is described in PCT Publication No. WO 00/18963 published on Apr. 6, 2000, the disclosure of which is hereby incorporated by reference.

The present invention encompasses functional fragments of the promoter sequences disclosed herein.

A “functional fragment” refer to a portion or subsequence of the promoter sequence of the present invention in which the ability to initiate transcription or drive gene expression (such as to produce a certain phenotype) is retained. Fragments can be obtained via methods such as site-directed mutagenesis and synthetic construction. As with the provided promoter sequences described herein, the functional fragments operate to promote the expression of an operably linked heterologous nucleotide sequence, forming a recombinant DNA construct (also, a chimeric gene). For example, the fragment can be used in the design of recombinant DNA constructs to produce the desired phenotype in a transformed plant. Recombinant DNA constructs can be designed for use in co-suppression or antisense by linking a promoter fragment in the appropriate orientation relative to a heterologous nucleotide sequence.

In an embodiment of the present invention, the promoters disclosed herein can be modified. Those skilled in the art can create promoters that have variations in the polynucleotide sequence. The polynucleotide sequence of the promoters of the present invention as shown in SEQ ID NOS: 1-6, may be modified or altered to enhance their control characteristics. As one of ordinary skill in the art will appreciate, modification or alteration of the promoter sequence can also be made without substantially affecting the promoter function. The methods are well known to those of skill in the art. Sequences can be modified, for example by insertion, deletion, or replacement of template sequences in a PCR-based DNA modification approach.

A “variant promoter”, as used herein, is the sequence of the promoter or the sequence of a functional fragment of a promoter containing changes in which one or more nucleotides of the original sequence is deleted, added, and/or substituted, while substantially maintaining promoter function. One or more base pairs can be inserted, deleted, or substituted internally to a promoter. In the case of a promoter fragment, variant promoters can include changes affecting the transcription of a minimal promoter to which it is operably linked. Variant promoters can be produced, for example, by standard DNA mutagenesis techniques or by chemically synthesizing the variant promoter or a portion thereof.

Methods for construction of chimeric and variant promoters of the present invention include, but are not limited to, combining control elements of different promoters or duplicating portions or regions of a promoter (see for example, U.S. Pat. No. 4,990,607; U.S. Pat. No. 5,110,732; and U.S. Pat. No. 5,097,025). Those of skill in the art are familiar with the standard resource materials that describe specific conditions and procedures for the construction, manipulation, and isolation of macromolecules (e.g., polynucleotide molecules and plasmids), as well as the generation of recombinant organisms and the screening and isolation of polynucleotide molecules.

In some aspects of the present invention, the promoter fragments can comprise at least about 20 contiguous nucleotides, or at least about 50 contiguous nucleotides, or at least about 75 contiguous nucleotides, or at least about 100 contiguous nucleotides, or at least about 150 contiguous nucleotides, or at least about 200 contiguous nucleotides of SEQ ID NO:1, SEQ ID NO:2, SEQ ID NO:3, SEQ ID NO:4, SEQ ID NO:5 or SEQ ID NO:6. In another aspect of the present invention, the promoter fragments can comprise at least about 250 contiguous nucleotides, or at least about 300 contiguous nucleotides, or at least about 350 contiguous nucleotides, or at least about 400 contiguous nucleotides, or at least about 450 contiguous nucleotides, or at least about 500 contiguous nucleotides, or at least about 550 contiguous nucleotides, or at least about 600 contiguous nucleotides, or at least about 650 contiguous nucleotides, or at least about 700 contiguous nucleotides, or at least about 750 contiguous nucleotides, or at least about 800 contiguous nucleotides, or at least about 850 contiguous nucleotides, or at least about 900 contiguous nucleotides, or at least about 950 contiguous nucleotides, or at least about 1000 contiguous nucleotides, or at least about 1050 contiguous nucleotides, or at least about 1100 contiguous nucleotides, or at least about 1150 contiguous nucleotides, or at least about 1200 contiguous nucleotides, or at least about 1250 contiguous nucleotides, or at least about 1300 contiguous nucleotides, or at least about 1350 contiguous nucleotides of SEQ ID NO:1. In another aspect, a promoter fragment is the nucleotide sequence set forth in SEQ ID NO:2, SEQ ID NO:3, SEQ ID NO:4, SEQ ID NO:5 or SEQ ID NO:6. The nucleotides of such fragments will usually comprise the TATA recognition sequence of the particular promoter sequence. Such fragments may be obtained by use of restriction enzymes to cleave the naturally occurring promoter nucleotide sequences disclosed herein, by synthesizing a nucleotide sequence from the naturally occurring promoter DNA sequence, or may be obtained through the use of PCR technology. See particularly, Mullis et al., Methods Enzymol. 155:335-350 (1987), and Higuchi, R. In PCR Technology: Principles and Applications for DNA Amplifications; Erlich, H. A., Ed.; Stockton Press Inc.: New York, 1989.

The terms “full complement” and “full-length complement” are used interchangeably herein, and refer to a complement of a given nucleotide sequence, wherein the complement and the nucleotide sequence consist of the same number of nucleotides and are 100% complementary.

The terms “substantially similar” and “corresponding substantially” as used herein refer to nucleic acid fragments wherein changes in one or more nucleotide bases do not affect the ability of the nucleic acid fragment to mediate gene expression or produce a certain phenotype. These terms also refer to modifications of the nucleic acid fragments of the instant invention such as deletion or insertion of one or more nucleotides that do not substantially alter the functional properties of the resulting nucleic acid fragment relative to the initial, unmodified fragment. It is therefore understood, as those skilled in the art will appreciate, that the invention encompasses more than the specific exemplary sequences.

The isolated promoter sequence of the present invention can be modified to provide a range of embryo-specific expression levels of the heterologous nucleotide sequence. Thus, less than the entire promoter regions may be utilized and the ability to drive expression of the coding sequence retained. However, it is recognized that expression levels of the mRNA may be decreased with deletions of portions of the promoter sequences. Likewise, the tissue-independent, constitutive nature of expression may be changed.

Modifications of the isolated promoter sequences of the present invention can provide for a range of embryo-specific expression of heterologous nucleotide sequences. Thus, they may be modified to be weak embryo-specific promoters or strong embryo-specific promoters. Generally, by “weak promoter” is intended a promoter that drives expression of a coding sequence at a low level. By “low level” is intended at levels about 1/10,000 transcripts to about 1/100,000 transcripts to about 1/500,000 transcripts. Conversely, a strong promoter drives expression of a coding sequence at high level, or at about 1/10 transcripts to about 1/100 transcripts to about 1/1,000 transcripts.

Moreover, the skilled artisan recognizes that substantially similar nucleic acid sequences encompassed by this invention are also defined by their ability to hybridize, under moderately stringent conditions (for example, 0.5×SSC, 0.1% SDS, 60° C.) with the sequences exemplified herein, or to any portion of the nucleotide sequences reported herein and which are functionally equivalent to the promoter of the invention. Estimates of such homology are provided by either DNA-DNA or DNA-RNA hybridization under conditions of stringency as is well understood by those skilled in the art (Hames and Higgins, Eds.; In Nucleic Acid Hybridisation; IRL Press: Oxford, U.K., 1985). Stringency conditions can be adjusted to screen for moderately similar fragments, such as homologous sequences from distantly related organisms, to highly similar fragments, such as genes that duplicate functional enzymes from closely related organisms. Post-hybridization washes partially determine stringency conditions. One set of conditions uses a series of washes starting with 6×SSC, 0.5% SDS at room temperature for 15 min, then repeated with 2×SSC, 0.5% SDS at 45° C. for 30 min, and then repeated twice with 0.2×SSC, 0.5% SDS at 50° C. for 30 min. Another set of stringent conditions uses higher temperatures in which the washes are identical to those above except for the temperature of the final two 30 min washes in 0.2×SSC, 0.5% SDS was increased to 60° C. Another set of highly stringent conditions uses two final washes in 0.1×SSC, 0.1% SDS at 65° C.

Preferred substantially similar nucleic acid sequences encompassed by this invention are those sequences that are 80% identical to the nucleic acid fragments reported herein or which are 80% identical to any portion of the nucleotide sequences reported herein. More preferred are nucleic acid fragments which are 90% identical to the nucleic acid sequences reported herein, or which are 90% identical to any portion of the nucleotide sequences reported herein. Most preferred are nucleic acid fragments which are 95% identical to the nucleic acid sequences reported herein, or which are 95% identical to any portion of the nucleotide sequences reported herein. It is well understood by one skilled in the art that many levels of sequence identity are useful in identifying related polynucleotide sequences. Useful examples of percent identities are those listed above, or also preferred is any integer percentage from 80% to 100%, such as 81%, 82%, 83%, 84%, 85%, 86%, 87%, 88%, 89%, 90%, 91%, 92%, 93%, 94%, 95%, 96%, 97%, 98 and 99%.

A “substantially homologous sequence” refers to variants of the disclosed sequences such as those that result from site-directed mutagenesis, as well as synthetically derived sequences. A substantially homologous sequence of the present invention also refers to those fragments of a particular promoter nucleotide sequence disclosed herein that operate to promote the embryo-specific expression of an operably linked heterologous nucleic acid fragment. These promoter fragments will comprise at least about 20 contiguous nucleotides, preferably at least about 50 contiguous nucleotides, more preferably at least about 75 contiguous nucleotides, even more preferably at least about 100 contiguous nucleotides of the particular promoter nucleotide sequence disclosed herein. The nucleotides of such fragments will usually comprise the TATA recognition sequence of the particular promoter sequence. Such fragments may be obtained by use of restriction enzymes to cleave the naturally occurring promoter nucleotide sequences disclosed herein; by synthesizing a nucleotide sequence from the naturally occurring promoter DNA sequence; or may be obtained through the use of PCR technology. See particularly, Mullis et al., Methods Enzymol. 155:335-350 (1987), and Higuchi, R. In PCR Technology: Principles and Applications for DNA Amplifications; Erlich, H. A., Ed.; Stockton Press Inc.: New York, 1989. Again, variants of these promoter fragments, such as those resulting from site-directed mutagenesis, are encompassed by the compositions of the present invention.

“Codon degeneracy” refers to divergence in the genetic code permitting variation of the nucleotide sequence without affecting the amino acid sequence of an encoded polypeptide. Accordingly, the instant invention relates to any nucleic acid fragment comprising a nucleotide sequence that encodes all or a substantial portion of the amino acid sequences set forth herein. The skilled artisan is well aware of the “codon-bias” exhibited by a specific host cell in usage of nucleotide codons to specify a given amino acid. Therefore, when synthesizing a nucleic acid fragment for improved expression in a host cell, it is desirable to design the nucleic acid fragment such that its frequency of codon usage approaches the frequency of preferred codon usage of the host cell.

Sequence alignments and percent similarity calculations may be determined using the Megalign program of the LASARGENE bioinformatics computing suite (DNASTAR Inc., Madison, Wis.) or using the AlignX program of the Vector NTI bioinformatics computing suite (Invitrogen). Multiple alignment of the sequences are performed using the Clustal method of alignment (Higgins and Sharp, CABIOS 5:151-153 (1989)) with the default parameters (GAP PENALTY=10, GAP LENGTH PENALTY=10). Default parameters for pairwise alignments and calculation of percent identity of protein sequences using the Clustal method are KTUPLE=1, GAP PENALTY=3, WINDOW=5 and DIAGONALS SAVED=5. For nucleic acids these parameters are GAP PENALTY=10, GAP LENGTH PENALTY=10, KTUPLE=2, GAP PENALTY=5, WINDOW-4 and DIAGONALS SAVED=4. A “substantial portion” of an amino acid or nucleotide sequence comprises enough of the amino acid sequence of a polypeptide or the nucleotide sequence of a gene to afford putative identification of that polypeptide or gene, either by manual evaluation of the sequence by one skilled in the art, or by computer-automated sequence comparison and identification using algorithms such as BLAST (Altschul, S. F. et al., J. Mol. Biol. 215:403-410 (1993)) and Gapped Blast (Altschul, S. F. et al., Nucleic Acids Res. 25:3389-3402 (1997)). BLASTN refers to a BLAST program that compares a nucleotide query sequence against a nucleotide sequence database.

“Gene” refers to a nucleic acid fragment that expresses a specific protein, including regulatory sequences preceding (5′ non-coding sequences) and following (3′ non-coding sequences) the coding sequence. “Native gene” refers to a gene as found in nature with its own regulatory sequences. “Chimeric gene” or “recombinant expression construct”, which are used interchangeably, refers to any gene that is not a native gene, comprising regulatory and coding sequences that are not found together in nature. Accordingly, a chimeric gene may comprise regulatory sequences and coding sequences that are derived from different sources, or regulatory sequences and coding sequences derived from the same source, but arranged in a manner different than that found in nature. “Endogenous gene” refers to a native gene in its natural location in the genome of an organism. A “foreign” gene refers to a gene not normally found in the host organism, but that is introduced into the host organism by gene transfer. Foreign genes can comprise native genes inserted into a non-native organism, or chimeric genes. A “transgene” is a gene that has been introduced into the genome by a transformation procedure.

“Coding sequence” refers to a DNA sequence which codes for a specific amino acid sequence. “Regulatory sequences” refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences may include, but are not limited to, promoters, translation leader sequences, introns, and polyadenylation recognition sequences.

An “intron” is an intervening sequence in a gene that is transcribed into RNA but is then excised in the process of generating the mature mRNA. The term is also used for the excised RNA sequences. An “exon” is a portion of the sequence of a gene that is transcribed and is found in the mature messenger RNA derived from the gene, but is not necessarily a part of the sequence that encodes the final gene product.

The “translation leader sequence” refers to a polynucleotide sequence located between the promoter sequence of a gene and the coding sequence. The translation leader sequence is present in the fully processed mRNA upstream of the translation start sequence. The translation leader sequence may affect processing of the primary transcript to mRNA, mRNA stability or translation efficiency. Examples of translation leader sequences have been described (Turner, R. and Foster, G. D., Molecular Biotechnology 3:225 (1995)).

The “3′ non-coding sequences” refer to DNA sequences located downstream of a coding sequence and include polyadenylation recognition sequences and other sequences encoding regulatory signals capable of affecting mRNA processing or gene expression. The polyadenylation signal is usually characterized by affecting the addition of polyadenylic acid tracts to the 3′ end of the mRNA precursor. The use of different 3′ non-coding sequences is exemplified by Ingelbrecht et al., Plant Cell 1:671-680 (1989).

“RNA transcript” refers to a product resulting from RNA polymerase-catalyzed transcription of a DNA sequence. When an RNA transcript is a perfect complementary copy of a DNA sequence, it is referred to as a primary transcript or it may be a RNA sequence derived from posttranscriptional processing of a primary transcript and is referred to as a mature RNA. “Messenger RNA” (“mRNA”) refers to RNA that is without introns and that can be translated into protein by the cell. “cDNA” refers to a DNA that is complementary to and synthesized from an mRNA template using the enzyme reverse transcriptase. The cDNA can be single-stranded or converted into the double-stranded by using the Klenow fragment of DNA polymerase I. “Sense” RNA refers to RNA transcript that includes mRNA and so can be translated into protein within a cell or in vitro. “Antisense RNA” refers to a RNA transcript that is complementary to all or part of a target primary transcript or mRNA and that blocks expression or transcripts accumulation of a target gene (U.S. Pat. No. 5,107,065). The complementarity of an antisense RNA may be with any part of the specific gene transcript, i.e. at the 5′ non-coding sequence, 3′ non-coding sequence, introns, or the coding sequence. “Functional RNA” refers to antisense RNA, ribozyme RNA, or other RNA that may not be translated but yet has an effect on cellular processes.

The term “operably linked” refers to the association of nucleic acid sequences on a single nucleic acid fragment so that the function of one is affected by the other. For example, a promoter is operably linked with a coding sequence when it is capable of affecting the expression of that coding sequence (i.e., that the coding sequence is under the transcriptional control of the promoter). Coding sequences can be operably linked to regulatory sequences in sense or antisense orientation.

The terms “initiate transcription”, “initiate expression”, “drive transcription”, and “drive expression” are used interchangeably herein and all refer to the primary function of a promoter. As detailed throughout this disclosure, a promoter is a non-coding genomic DNA sequence, usually upstream (5′) to the relevant coding sequence, and its primary function is to act as a binding site for RNA polymerase and initiate transcription by the RNA polymerase. Additionally, there is “expression” of RNA, including functional RNA, or the expression of polypeptide for operably linked encoding nucleotide sequences, as the transcribed RNA ultimately is translated into the corresponding polypeptide.

The term “expression”, as used herein, refers to the production of a functional end-product e.g., an mRNA or a protein (precursor or mature).

The term “expression cassette” as used herein, refers to a discrete nucleic acid fragment into which a nucleic acid sequence or fragment can be moved.

Expression or overexpression of a gene involves transcription of the gene and translation of the mRNA into a precursor or mature protein. “Antisense inhibition” refers to the production of antisense RNA transcripts capable of suppressing the expression of the target protein. “Overexpression” refers to the production of a gene product in transgenic organisms that exceeds levels of production in normal or non-transformed organisms. “Co-suppression” refers to the production of sense RNA transcripts capable of suppressing the expression or transcript accumulation of identical or substantially similar foreign or endogenous genes (U.S. Pat. No. 5,231,020). The mechanism of co-suppression may be at the DNA level (such as DNA methylation), at the transcriptional level, or at posttranscriptional level.

Co-suppression constructs in plants previously have been designed by focusing on overexpression of a nucleic acid sequence having homology to an endogenous mRNA, in the sense orientation, which results in the reduction of all RNA having homology to the overexpressed sequence (see Vaucheret et al., Plant J. 16:651-659 (1998); and Gura, Nature 404:804-808 (2000)). The overall efficiency of this phenomenon is low, and the extent of the RNA reduction is widely variable. Recent work has described the use of “hairpin” structures that incorporate all, or part, of an mRNA encoding sequence in a complementary orientation that results in a potential “stem-loop” structure for the expressed RNA (PCT Publication No. WO 99/53050 published on Oct. 21, 1999; and PCT Publication No. WO 02/00904 published on Jan. 3, 2002). This increases the frequency of co-suppression in the recovered transgenic plants. Another variation describes the use of plant viral sequences to direct the suppression, or “silencing”, of proximal mRNA encoding sequences (PCT Publication No. WO 98/36083 published on Aug. 20, 1998). Genetic and molecular evidences have been obtained suggesting that dsRNA mediated mRNA cleavage may have been the conserved mechanism underlying these gene silencing phenomena (Elmayan et al., Plant Cell 10:1747-1757 (1998); Galun, In Vitro Cell. Dev. Biol. Plant 41(2):113-123 (2005); Pickford et al, Cell. Mol. Life Sci. 60(5):871-882 (2003)).

As stated herein, “suppression” refers to a reduction of the level of enzyme activity or protein functionality (e.g., a phenotype associated with a protein) detectable in a transgenic plant when compared to the level of enzyme activity or protein functionality detectable in a non-transgenic or wild type plant with the native enzyme or protein. The level of enzyme activity in a plant with the native enzyme is referred to herein as “wild type” activity. The level of protein functionality in a plant with the native protein is referred to herein as “wild type” functionality. The term “suppression” includes lower, reduce, decline, decrease, inhibit, eliminate and prevent. This reduction may be due to a decrease in translation of the native mRNA into an active enzyme or functional protein. It may also be due to the transcription of the native DNA into decreased amounts of mRNA and/or to rapid degradation of the native mRNA. The term “native enzyme” refers to an enzyme that is produced naturally in a non-transgenic or wild type cell. The terms “non-transgenic” and “wild type” are used interchangeably herein.

“Altering expression” refers to the production of gene product(s) in transgenic organisms in amounts or proportions that differ significantly from the amount of the gene product(s) produced by the corresponding wild-type organisms (i.e., expression is increased or decreased).

“Transformation” as used herein refers to both stable transformation and transient transformation.

“Stable transformation” refers to the introduction of a nucleic acid fragment into a genome of a host organism resulting in genetically stable inheritance. Once stably transformed, the nucleic acid fragment is stably integrated in the genome of the host organism and any subsequent generation. Host organisms containing the transformed nucleic acid fragments are referred to as “transgenic” organisms.

“Transient transformation” refers to the introduction of a nucleic acid fragment into the nucleus, or DNA-containing organelle, of a host organism resulting in gene expression without genetically stable inheritance.

The term “introduced” means providing a nucleic acid (e.g., expression construct) or protein into a cell. Introduced includes reference to the incorporation of a nucleic acid into a eukaryotic or prokaryotic cell where the nucleic acid may be incorporated into the genome of the cell, and includes reference to the transient provision of a nucleic acid or protein to the cell. Introduced includes reference to stable or transient transformation methods, as well as sexually crossing. Thus, “introduced” in the context of inserting a nucleic acid fragment (e.g., a recombinant DNA construct/expression construct) into a cell, means “transfection” or “transformation” or “transduction” and includes reference to the incorporation of a nucleic acid fragment into a eukaryotic or prokaryotic cell where the nucleic acid fragment may be incorporated into the genome of the cell (e.g., chromosome, plasmid, plastid or mitochondrial DNA), converted into an autonomous replicon, or transiently expressed (e.g., transfected mRNA).

“Transgenic” refers to any cell, cell line, callus, tissue, plant part or plant, the genome of which has been altered by the presence of a heterologous nucleic acid, such as a recombinant DNA construct, including those initial transgenic events as well as those created by sexual crosses or asexual propagation from the initial transgenic event. The term “transgenic” as used herein does not encompass the alteration of the genome (chromosomal or extra-chromosomal) by conventional plant breeding methods or by naturally occurring events such as random cross-fertilization, non-recombinant viral infection, non-recombinant bacterial transformation, non-recombinant transposition, or spontaneous mutation.

“Genome” as it applies to plant cells encompasses not only chromosomal DNA found within the nucleus, but organelle DNA found within subcellular components (e.g., mitochondrial, plastid) of the cell.

“Plant” includes reference to whole plants, plant organs, plant tissues, seeds and plant cells and progeny of same. Plant cells include, without limitation, cells from seeds, suspension cultures, embryos, meristematic regions, callus tissue, leaves, roots, shoots, gametophytes, sporophytes, pollen, and microspores.

The terms “monocot” and “monocotyledonous plant” are used interchangeably herein. A monocot of the current invention includes the Gramineae.

The terms “dicot” and “dicotyledonous plant” are used interchangeably herein. A dicot of the current invention includes the following families: Brassicaceae, Leguminosae, and Solanaceae.

“Progeny” comprises any subsequent generation of a plant.

“Transgenic plant” includes reference to a plant which comprises within its genome a heterologous polynucleotide. For example, the heterologous polynucleotide is stably integrated within the genome such that the polynucleotide is passed on to successive generations. The heterologous polynucleotide may be integrated into the genome alone or as part of a recombinant DNA construct.

“Transient expression” refers to the temporary expression of often reporter genes such as β-glucuronidase (GUS), fluorescent protein genes ZS-GREEN1, ZS-YELLOW1 N1, AM-CYAN1, DS-RED in selected certain cell types of the host organism in which the transgenic gene is introduced temporally by a transformation method. The transformed materials of the host organism are subsequently discarded after the transient gene expression assay.

Standard recombinant DNA and molecular cloning techniques used herein are well known in the art and are described more fully in Sambrook, J. et al., In Molecular Cloning: A Laboratory Manual; 2^{nd} ed.; Cold Spring Harbor Laboratory Press: Cold Spring Harbor, N.Y., 1989 (hereinafter “Sambrook et al., 1989”) or Ausubel, F. M., Brent, R., Kingston, R. E., Moore, D. D., Seidman, J. G., Smith, J. A. and Struhl, K., Eds.; In Current Protocols in Molecular Biology; John Wiley and Sons: New York, 1990 (hereinafter “Ausubel et al., 1990”).

“PCR” or “Polymerase Chain Reaction” is a technique for the synthesis of large quantities of specific DNA segments, consisting of a series of repetitive cycles (Perkin Elmer Cetus Instruments, Norwalk, Conn.). Typically, the double stranded DNA is heat denatured, the two primers complementary to the 3′ boundaries of the target segment are annealed at low temperature and then extended at an intermediate temperature. One set of these three consecutive steps comprises a cycle.

The terms “plasmid”, “vector” and “cassette” refer to an extra chromosomal element often carrying genes that are not part of the central metabolism of the cell, and usually in the form of circular double-stranded DNA fragments. Such elements may be autonomously replicating sequences, genome integrating sequences, phage or nucleotide sequences, linear or circular, of a single- or double-stranded DNA or RNA, derived from any source, in which a number of nucleotide sequences have been joined or recombined into a unique construction which is capable of introducing a promoter fragment and DNA sequence for a selected gene product along with appropriate 3′ untranslated sequence into a cell.

The term “recombinant DNA construct” or “recombinant expression construct” is used interchangeably and refers to a discrete polynucleotide into which a nucleic acid sequence or fragment can be moved. Preferably, it is a plasmid vector or a fragment thereof comprising the promoters of the present invention. The choice of plasmid vector is dependent upon the method that will be used to transform host plants. The skilled artisan is well aware of the genetic elements that must be present on the plasmid vector in order to successfully transform, select and propagate host cells containing the chimeric gene. The skilled artisan will also recognize that different independent transformation events will result in different levels and patterns of expression (Jones et al., EMBO J. 4:2411-2418 (1985); De Almeida et al., Mol. Gen. Genetics 218:78-86 (1989)), and thus that multiple events must be screened in order to obtain lines displaying the desired expression level and pattern. Such screening may be accomplished by PCR and Southern analysis of DNA, RT-PCR and Northern analysis of mRNA expression, Western analysis of protein expression, or phenotypic analysis.

Various changes in phenotype are of interest including, but not limited to, modifying the fatty acid composition in a plant, altering the amino acid content of a plant, altering a plant's pathogen defense mechanism, and the like. These results can be achieved by providing expression of heterologous products or increased expression of endogenous products in plants. Alternatively, the results can be achieved by providing for a reduction of expression of one or more endogenous products, particularly enzymes or cofactors in the plant. These changes result in a change in phenotype of the transformed plant.

Genes of interest are reflective of the commercial markets and interests of those involved in the development of the crop. Crops and markets of interest change, and as developing nations open up world markets, new crops and technologies will emerge also. In addition, as our understanding of agronomic characteristics and traits such as yield and heterosis increase, the choice of genes for transformation will change accordingly. General categories of genes of interest include, but are not limited to, those genes involved in information, such as zinc fingers, those involved in communication, such as kinases, and those involved in housekeeping, such as heat shock proteins. More specific categories of transgenes, for example, include, but are not limited to, genes encoding important traits for agronomics, insect resistance, disease resistance, herbicide resistance, sterility, grain or seed characteristics, and commercial products. Genes of interest include, generally, those involved in oil, starch, carbohydrate, or nutrient metabolism as well as those affecting seed size, plant development, plant growth regulation, and yield improvement. Plant development and growth regulation also refer to the development and growth regulation of various parts of a plant, such as the flower, seed, root, leaf and shoot.

Other commercially desirable traits are genes and proteins conferring cold, heat, salt, and drought resistance.

Disease and/or insect resistance genes may encode resistance to pests that have great yield drag such as for example, anthracnose, soybean mosaic virus, soybean cyst nematode, root-knot nematode, brown leaf spot, Downy mildew, purple seed stain, seed decay and seedling diseases caused commonly by the fungi—Pythium sp., Phytophthora sp., Rhizoctonia sp., Diaporthe sp. Bacterial blight caused by the bacterium Pseudomonas syringae pv. Glycinea. Genes conferring insect resistance include, for example, Bacillus thuringiensis toxic protein genes (U.S. Pat. Nos. 5,366,892; 5,747,450; 5,737,514; 5,723,756; 5,593,881; and Geiser et al (1986) Gene 48:109); lectins (Van Damme et al. (1994) Plant Mol. Biol. 24:825); and the like.

Herbicide resistance traits may include genes coding for resistance to herbicides that act to inhibit the action of acetolactate synthase (ALS), in particular the sulfonylurea-type herbicides (e.g., the acetolactate synthase ALS gene containing mutations leading to such resistance, in particular the S4 and/or HRA mutations). The ALS-gene mutants encode resistance to the herbicide chlorsulfuron. Glyphosate acetyl transferase (GAT) is an N-acetyltransferase from Bacillus licheniformis that was optimized by gene shuffling for acetylation of the broad spectrum herbicide, glyphosate, forming the basis of a novel mechanism of glyphosate tolerance in transgenic plants (Castle et al. (2004) Science 304, 1151-1154).

Antibiotic resistance genes include, for example, neomycin phosphotransferase (npt) and hygromycin phosphotransferase (hpt). Two neomycin phosphotransferase genes are used in selection of transformed organisms: the neomycin phosphotransferase I (nptI) gene and the neomycin phosphotransferase II (nptII) gene. The second one is more widely used. It was initially isolated from the transposon Tn5 that was present in the bacterium strain Escherichia coli K12. The gene codes for the aminoglycoside 3′-phosphotransferase (denoted aph(3′)-II or NPTII) enzyme, which inactivates by phosphorylation a range of aminoglycoside antibiotics such as kanamycin, neomycin, geneticin and paroromycin. NPTII is widely used as a selectable marker for plant transformation. It is also used in gene expression and regulation studies in different organisms in part because N-terminal fusions can be constructed that retain enzyme activity. NPTII protein activity can be detected by enzymatic assay. In other detection methods, the modified substrates, the phosphorylated antibiotics, are detected by thin-layer chromatography, dot-blot analysis or polyacrylamide gel electrophoresis. Plants such as maize, cotton, tobacco, Arabidopsis, flax, soybean and many others have been successfully transformed with the nptII gene.

The hygromycin phosphotransferase (denoted hpt, hph or aphIV) gene was originally derived from Escherichia coli. The gene codes for hygromycin phosphotransferase (HPT), which detoxifies the aminocyclitol antibiotic hygromycin B. A large number of plants have been transformed with the hpt gene and hygromycin B has proved very effective in the selection of a wide range of plants, including monocotyledonous. Most plants exhibit higher sensitivity to hygromycin B than to kanamycin, for instance cereals. Likewise, the hpt gene is used widely in selection of transformed mammalian cells. The sequence of the hpt gene has been modified for its use in plant transformation. Deletions and substitutions of amino acid residues close to the carboxy (C)-terminus of the enzyme have increased the level of resistance in certain plants, such as tobacco. At the same time, the hydrophilic C-terminus of the enzyme has been maintained and may be essential for the strong activity of HPT. HPT activity can be checked using an enzymatic assay. A non-destructive callus induction test can be used to verify hygromycin resistance.

Genes involved in plant growth and development have been identified in plants. One such gene, which is involved in cytokinin biosynthesis, is isopentenyl transferase (IPT). Cytokinin plays a critical role in plant growth and development by stimulating cell division and cell differentiation (Sun et al. (2003), Plant Physiol. 131: 167-176).

Calcium-dependent protein kinases (CDPK), a family of serine-threonine kinase found primarily in the plant kingdom, are likely to function as sensor molecules in calcium-mediated signaling pathways. Calcium ions are important second messengers during plant growth and development (Harper et al. Science 252, 951-954 (1993); Roberts et al. Curr. Opin. Cell Biol. 5, 242-246 (1993); Roberts et al. Annu. Rev. Plant Mol. Biol. 43, 375-414 (1992)).

Nematode responsive protein (NRP) is produced by soybean upon the infection of soybean cyst nematode. NRP has homology to a taste-modifying glycoprotein miraculin and the NF34 protein involved in tumor formation and hyper response induction. NRP is believed to function as a defense-inducer in response to nematode infection (Tenhaken et al. BMC Bioinformatics 6:169 (2005)).

The quality of seeds and grains is reflected in traits such as levels and types of fatty acids or oils, saturated and unsaturated, quality and quantity of essential amino acids, and levels of carbohydrates. Therefore, commercial traits can also be encoded on a gene or genes that could increase for example methionine and cysteine, two sulfur containing amino acids that are present in low amounts in soybeans. Cystathionine gamma synthase (CGS) and serine acetyl transferase (SAT) are proteins involved in the synthesis of methionine and cysteine, respectively.

Other commercial traits can encode genes to increase for example monounsaturated fatty acids, such as oleic acid, in oil seeds. Soybean oil for example contains high levels of polyunsaturated fatty acids and is more prone to oxidation than oils with higher levels of monounsaturated and saturated fatty acids. High oleic soybean seeds can be prepared by recombinant manipulation of the activity of oleoyl 12-desaturase (Fad2). High oleic soybean oil can be used in applications that require a high degree of oxidative stability, such as cooking for a long period of time at an elevated temperature.

Raffinose saccharides accumulate in significant quantities in the edible portion of many economically significant crop species, such as soybean (Glycine max L. Merrill), sugar beet (Beta vulgaris), cotton (Gossypium hirsutum L.), canola (Brassica sp.) and all of the major edible leguminous crops including beans (Phaseolus sp.), chick pea (Cicer arietinum), cowpea (Vigna unguiculata), mung bean (Vigna radiata), peas (Pisum sativum), lentil (Lens culinaris) and lupine (Lupinus sp.). Although abundant in many species, raffinose saccharides are an obstacle to the efficient utilization of some economically important crop species.

Down regulation of the expression of the enzymes involved in raffinose saccharide synthesis, such as galactinol synthase for example, would be a desirable trait.

In certain embodiments, the present invention contemplates the transformation of a recipient cell with more than one advantageous transgene. Two or more transgenes can be supplied in a single transformation event using either distinct transgene-encoding vectors, or a single vector incorporating two or more gene coding sequences. Any two or more transgenes of any description, such as those conferring herbicide, insect, disease (viral, bacterial, fungal, and nematode) or drought resistance, oil quantity and quality, or those increasing yield or nutritional quality may be employed as desired.

The Bowman-Birk inhibitor (BBI) is a small water-soluble protein present in soybean and almost all monocotyledonous and dicotyledonous seeds. BBI can withstand boiling water temperature for 10 minutes, is resistant to the pH range and proteolytic enzymes of the gastrointestinal tract, and not allergenic. BBI reduces the proteolytic activities of trypsin, chymotrypsin, elastase, cathepsin G, and chymase, serine protease-dependent matrix metalloproteinases, and some protein kinases (Losso, Critical Rev. Food Sci. Nutr. 48:94-118 (2008)). There are ten Bowman-Birk protease isoinhibitors identified immunologically in soybean and purified and characterized biochemically (Tan-Wilson et al., J. Agric. Food Chem. 33:389-393 (1985) and 35:974-981 (1987)). Many members of the soybean BBI multigene family have also been cloned (Baek et al., Biosci. Biotechnol. Biochem. 58:843-846 (1994); Deshimaru et al., Biosci. Biotechnol. Biochem. 68:1279-1286 (2004)), including the Glycine soja BBI isoinhibitor D which is identical to the BBI3 cDNA sequence SEQ ID NO:1 in this invention (Yoshimi et al., Biosci. Biotechnol. Biochem. 68:1279-1286 (2004)). It is demonstrated herein that the soybean Bowman-Birk inhibitor gene promoter BBI3 can, in fact, be used as an embryo-specific promoter to drive efficient expression of transgenes, and that such promoter can be isolated and used by one skilled in the art.

This invention concerns an isolated nucleotide sequence comprising an embryo-specific BBI gene promoter BBI3. This invention also concerns an isolated nucleotide sequence comprising a promoter wherein said promoter consists essentially of the nucleotide sequence set forth in SEQ ID NO:1, or an isolated polynucleotide comprising a promoter wherein said promoter comprises the nucleotide sequence set forth in SEQ ID NOs: 1, 2, 3, 4, 5, or 6, or a functional fragment of SEQ ID NOs: 1, 2, 3, 4, 5, or 6.

The expression patterns of BBI3 gene and its promoter are set forth in Examples 1, 2, 7, and 8.

The promoter activity of the soybean genomic DNA fragment SEQ ID NO:1 upstream of the BBI3 protein coding sequence was assessed by linking the fragment to a yellow fluorescence reporter gene, ZS-YELLOW1 N1 (YFP) (Matz et al, Nat. Biotechnol. 17:969-973 (1999)), transforming the promoter:YFP expression cassette into soybean, and analyzing YFP expression in various cell types of the transgenic plants (see Example 7 and 8). YFP expression was primarily detected in developing embryos and pods. These results indicated that the nucleotide fragment contained an embryo-specific promoter.

It is clear from the disclosure set forth herein that one of ordinary skill in the art could perform the following procedure:

1) operably linking the nucleic acid fragment containing the BBI3 promoter sequence to a suitable reporter gene; there are a variety of reporter genes that are well known to those skilled in the art, including the bacterial GUS gene, the firefly luciferase gene, and the cyan, green, red, and yellow fluorescent protein genes; any gene for which an easy and reliable assay is available can serve as the reporter gene.

2) transforming a chimeric BBI3 promoter:reporter gene expression cassette into an appropriate plant for expression of the promoter. There are a variety of appropriate plants which can be used as a host for transformation that are well known to those skilled in the art, including the dicots, Arabidopsis, tobacco, soybean, oilseed rape, peanut, sunflower, safflower, cotton, tomato, potato, cocoa and the monocots, corn, wheat, rice, barley and palm.

3) testing for expression of the BBI3 promoter in various cell types of transgenic plant tissues, e.g., leaves, roots, flowers, seeds, transformed with the chimeric BBI3 promoter reporter gene expression cassette by assaying for expression of the reporter gene product.

In another aspect, this invention concerns a recombinant DNA construct comprising at least one heterologous nucleic acid fragment operably linked to any promoter, or combination of promoter elements, of the present invention. Recombinant DNA constructs can be constructed by operably linking the nucleic acid fragment of the invention BBI3 promoter or a fragment that is substantially similar and functionally equivalent to any portion of the nucleotide sequence set forth in SEQ ID NOs:1, 2, 3, 4, 5, or 6 to a heterologous nucleic acid fragment. Any heterologous nucleic acid fragment can be used to practice the invention. The selection will depend upon the desired application or phenotype to be achieved. The various nucleic acid sequences can be manipulated so as to provide for the nucleic acid sequences in the proper orientation. It is believed that various combinations of promoter elements as described herein may be useful in practicing the present invention.

In another aspect, this invention concerns a recombinant DNA construct comprising at least one acetolactate synthase (ALS) nucleic acid fragment operably linked to BBI3 promoter, or combination of promoter elements, of the present invention. The acetolactate synthase gene is involved in the biosynthesis of branched chain amino acids in plants and is the site of action of several herbicides including sulfonyl urea. Expression of a mutated acetolactate synthase gene encoding a protein that can no longer bind the herbicide will enable the transgenic plants to be resistant to the herbicide (U.S. Pat. No. 5,605,011, U.S. Pat. No. 5,378,824). The mutated acetolactate synthase gene (HRA, High Resistance Allele) is also widely used in plant transformation to select transgenic plants.

In another embodiment, this invention concerns host cells comprising either the recombinant DNA constructs of the invention as described herein or isolated polynucleotides of the invention as described herein. Examples of host cells which can be used to practice the invention include, but are not limited to, yeast, bacteria, and plants.

Plasmid vectors comprising the instant recombinant expression construct can be constructed. The choice of plasmid vector is dependent upon the method that will be used to transform host cells. The skilled artisan is well aware of the genetic elements that must be present on the plasmid vector in order to successfully transform, select and propagate host cells containing the chimeric gene.

Methods for transforming dicots, primarily by use of Agrobacterium tumefaciens, and obtaining transgenic plants have been published, among others, for cotton (U.S. Pat. No. 5,004,863, U.S. Pat. No. 5,159,135); soybean (U.S. Pat. No. 5,569,834, U.S. Pat. No. 5,416,011); Brassica (U.S. Pat. No. 5,463,174); peanut (Cheng et al., Plant Cell Rep. 15:653-657 (1996), McKently et al., Plant Cell Rep. 14:699-703 (1995)); papaya (Ling et al., Bio/technology 9:752-758 (1991)); and pea (Grant et al., Plant Cell Rep. 15:254-258 (1995)). For a review of other commonly used methods of plant transformation see Newell, C. A., Mol. Biotechnol. 16:53-65 (2000). One of these methods of transformation uses Agrobacterium rhizogenes (Tepfler, M. and Casse-Delbart, F., Microbiol. Sci. 4:24-28 (1987)). Transformation of soybeans using direct delivery of DNA has been published using PEG fusion (PCT Publication No. WO 92/17598), electroporation (Chowrira et al., Mol. Biotechnol. 3:17-23 (1995); Christou et al., Proc. Natl. Acad. Sci. U.S.A. 84:3962-3966 (1987)), microinjection, or particle bombardment (McCabe et al., Biotechnology 6:923-926 (1988); Christou et al., Plant Physiol. 87:671-674 (1988)).

There are a variety of methods for the regeneration of plants from plant tissues. The particular method of regeneration will depend on the starting plant tissue and the particular plant species to be regenerated. The regeneration, development and cultivation of plants from single plant protoplast transformants or from various transformed explants is well known in the art (Weissbach and Weissbach, Eds.; In Methods for Plant Molecular Biology; Academic Press. Inc.: San Diego, Calif., 1988). This regeneration and growth process typically includes the steps of selection of transformed cells, culturing those individualized cells through the usual stages of embryonic development or through the rooted plantlet stage. Transgenic embryos and seeds are similarly regenerated. The resulting transgenic rooted shoots are thereafter planted in an appropriate plant growth medium such as soil. Preferably, the regenerated plants are self-pollinated to provide homozygous transgenic plants. Otherwise, pollen obtained from the regenerated plants is crossed to seed-grown plants of agronomically important lines. Conversely, pollen from plants of these important lines is used to pollinate regenerated plants. A transgenic plant of the present invention containing a desired polypeptide is cultivated using methods well known to one skilled in the art.

In addition to the above discussed procedures, practitioners are familiar with the standard resource materials which describe specific conditions and procedures for the construction, manipulation and isolation of macromolecules (e.g., DNA molecules, plasmids, etc.), generation of recombinant DNA fragments and recombinant expression constructs and the screening and isolating of clones, (see for example, Sambrook, J. et al., In Molecular Cloning: A Laboratory Manual; 2^{nd} ed.; Cold Spring Harbor Laboratory Press: Cold Spring Harbor, N.Y., 1989; Maliga et al., In Methods in Plant Molecular Biology; Cold Spring Harbor Press, 1995; Birren et al., In Genome Analysis: Detecting Genes, 1; Cold Spring Harbor: New York, 1998; Birren et al., In Genome Analysis: Analyzing DNA, 2; Cold Spring Harbor: New York, 1998; Clark, Ed., In Plant Molecular Biology: A Laboratory Manual; Springer: New York, 1997).

The skilled artisan will also recognize that different independent transformation events will result in different levels and patterns of expression of the chimeric genes (Jones et al., EMBO J. 4:2411-2418 (1985); De Almeida et al., Mol. Gen. Genetics 218:78-86 (1989)). Thus, multiple events must be screened in order to obtain lines displaying the desired expression level and pattern. Such screening may be accomplished by Northern analysis of mRNA expression, Western analysis of protein expression, or phenotypic analysis. Also of interest are seeds obtained from transformed plants displaying the desired gene expression profile.

Tissue-specific expression of chimeric genes in embryo cells makes the BBI3 promoter of the instant invention especially useful when embryo-specific or seed specific expression of a target heterologous nucleic acid fragment is required.

Another general application of the BBI3 promoter of the invention is to construct chimeric genes that can be used to reduce expression of at least one heterologous nucleic acid fragment in a plant cell. To accomplish this, a chimeric gene designed for gene silencing of a heterologous nucleic acid fragment can be constructed by linking the fragment to the BBI3 promoter of the present invention. (See U.S. Pat. No. 5,231,020, and PCT Publication No. WO 99/53050 published on Oct. 21, 1999, PCT Publication No. WO 02/00904 published on Jan. 3, 2002, and PCT Publication No. WO 98/36083 published on Aug. 20, 1998, for methodology to block plant gene expression via cosuppression.) Alternatively, a chimeric gene designed to express antisense RNA for a heterologous nucleic acid fragment can be constructed by linking the fragment in reverse orientation to the BBI3 promoter of the present invention. (See U.S. Pat. No. 5,107,065 for methodology to block plant gene expression via antisense RNA.) Either the cosuppression or antisense chimeric gene can be introduced into plants via transformation. Transformants wherein expression of the heterologous nucleic acid fragment is decreased or eliminated are then selected.

This invention also concerns a method of altering (increasing or decreasing) the expression of at least one heterologous nucleic acid fragment in a plant cell which comprises:
(a) transforming a plant cell with the recombinant expression construct described herein;
(b) growing fertile mature plants from the transformed plant cell of step (a);
(c) selecting plants containing a transformed plant cell wherein the expression of the heterologous nucleic acid fragment is increased or decreased.

Transformation and selection can be accomplished using methods well-known to those skilled in the art including, but not limited to, the methods described herein.

Non-limiting examples of methods and compositions disclosed herein are as follows:

1. An isolated polynucleotide comprising a promoter region of the BBI3 Glycine max gene as set forth in SEQ ID NO:1, wherein said promoter comprises a deletion at the 5′-terminus of 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, 200, 201, 202, 203, 204, 205, 206, 207, 208, 209, 210, 211, 212, 213, 214, 215, 216, 217, 218, 219, 220, 221, 222, 223, 224, 225, 226, 227, 228, 229, 230, 231, 232, 233, 234, 235, 236, 237, 238, 239, 240, 241, 242, 243, 244, 245, 246, 247, 248, 249, 250, 251, 252, 253, 254, 255, 256, 257, 258, 259, 260, 261, 262, 263, 264, 265, 266, 267, 268, 269, 270, 271, 272, 273, 274, 275, 276, 277, 278, 279, 280, 281, 282, 283, 284, 285, 286, 287, 288, 289, 290, 291, 292, 293, 294, 295, 296, 297, 298, 299, 300, 301, 302, 303, 304, 305, 306, 307, 308, 309, 310, 311, 312, 313, 314, 315, 316, 317, 318, 319, 320, 321, 322, 323, 324, 325, 326, 327, 328, 329, 330, 331, 332, 333, 334, 335, 336, 337, 338, 339, 340, 341, 342, 343, 344, 345, 346, 347, 348, 349, 350, 351, 352, 353, 354, 355, 356, 357, 358, 359, 360, 361, 362, 363, 364, 365, 366, 367, 368, 369, 370, 371, 372, 373, 374, 375, 376, 377, 378, 379, 380, 381, 382, 383, 384, 385, 386, 387, 388, 389, 390, 391, 392, 393, 394, 395, 396, 397, 398, 399, 400, 401, 402, 403, 404, 405, 406, 407, 408, 409, 410, 411, 412, 413, 414, 415, 416, 417, 418, 419, 420, 421, 422, 423, 424, 425, 426, 427, 428, 429, 430, 431, 432, 433, 434, 435, 436, 437, 438, 439, 440, 441, 442, 443, 444, 445, 446, 447, 448, 449, 450, 451, 452, 453, 454, 455, 456, 457, 458, 459, 460, 461, 462, 463, 464, 465, 466, 467, 468, 469, 470, 471, 472, 473, 474, 475, 476, 477, 478, 479, 480, 481, 482, 483, 484, 485, 486, 487, 488, 489, 490, 491, 492, 493, 494, 495, 496, 497, 498, 499, 500, 501, 502, 503, 504, 505, 506, 507, 508, 509, 510, 511, 512, 513, 514, 515, 516, 517, 518, 519, 520, 521, 522, 523, 524, 525, 526, 527, 528, 529, 530, 531, 532, 533, 534, 535, 536, 537, 538, 539, 540, 541, 542, 543, 544, 545, 546, 547, 548, 549, 550, 551, 552, 553, 554, 555, 556, 557, 558, 559, 560, 561, 562, 563, 564, 565, 566, 567, 568, 569, 570, 571, 572, 573, 574, 575, 576, 577, 578, 579, 580, 581, 582, 583, 584, 585, 586, 587, 588, 589, 590, 591, 592, 593, 594, 595, 596, 597, 598, 599, 600, 601, 602, 603, 604, 605, 606, 607, 608, 609, 610, 611, 612, 613, 614, 615, 616, 617, 618, 619, 620, 621, 622, 623, 624, 625, 626, 627, 628, 629, 630, 631, 632, 633, 634, 635, 636, 637, 638, 639, 640, 641, 642, 643, 644, 645, 646, 647, 648, 649, 650, 651, 652, 653, 654, 655, 656, 657, 658, 659, 660, 661, 662, 663, 664, 665, 666, 667, 668, 669, 670, 671, 672, 673, 674, 675, 676, 677, 678, 679, 680, 681, 682, 683, 684, 685, 686, 687, 688, 689, 690, 691, 692, 693, 694, 695, 696, 697, 698, 699, 700, 701, 702, 703, 704, 705, 706, 707, 708, 709, 710, 711, 712, 713, 714, 715, 716, 717, 718, 719, 720, 721, 722, 723, 724, 725, 726, 727, 728, 729, 730, 731, 732, 733, 734, 735, 736, 737, 738, 739, 740, 741, 742, 743, 744, 745, 746, 747, 748, 749, 750, 751, 752, 753, 754, 755, 756, 757, 758, 759, 760, 761, 762, 763, 764, 765, 766, 767, 768, 769, 770, 771, 772, 773, 774, 775, 776, 777, 778, 779, 780, 781, 782, 783, 784, 785, 786, 787, 788, 789, 790, 791, 792, 793, 794, 795, 796, 797, 798, 799, 800, 801, 802, 803, 804, 805, 806, 807, 808, 809, 810, 811, 812, 813, 814, 815, 816, 817, 818, 819, 820, 821, 822, 823, 824, 825, 826, 827, 828, 829, 830, 831, 832, 833, 834, 835, 836, 837, 838, 839, 840, 841, 842, 843, 844, 845, 846, 847, 848, 849, 850, 851, 852, 853, 854, 855, 856, 857, 858, 859, 860, 861, 862, 863, 864, 865, 866, 867, 868, 869, 870, 871, 872, 873, 874, 875, 876, 877, 878, 879, 880, 881, 882, 883, 884, 885, 886, 887, 888, 889, 890, 891, 892, 893, 894, 895, 896, 897, 898, 899, 900, 901, 902, 903, 904, 905, 906, 907, 908, 909, 910, 911, 912, 913, 914, 915, 916, 917, 918, 919, 920, 921, 922, 923, 924, 925, 926, 927, 928, 929, 930, 931, 932, 933, 934, 935, 936, 937, 938, 939, 940, 941, 942, 943, 944, 945, 946, 947, 948, 949, 950, 951, 952, 953, 954, 955, 956, 957, 958, 959, 960, 961, 962, 963, 964, 965, 966, 967, 968, 969, 970, 971, 972, 973, 974, 975, 976, 977, 978, 979, 980, 981, 982, 983, 984, 985, 986, 987, 988, 989, 990, 991, 992, 993, 994, 995, 996, 997, 998, 999, 1000, 1001, 1002, 1003, 1004, 1005, 1006, 1007, 1008, 1009, 1010, 1011, 1012, 1013, 1014, 1015, 1016, 1017, 1018, 1019, 1020, 1021, 1022, 1023, 1024, 1025, 1026, 1027, 1028, 1029, 1030, 1031, 1032, 1033, 1034, 1035, 1036, 1037, 1038, 1039, 1040, 1041, 1042, 1043, 1044, 1045, 1046, 1047, 1048, 1049, 1050, 1051, 1052, 1053, 1054, 1055, 1056, 1057, 1058, 1059, 1060, 1061, 1062, 1063, 1064, 1065, 1066, 1067, 1068, 1069, 1070, 1071, 1072, 1073, 1074, 1075, 1076, 1077, 1078, 1079, 1080, 1081, 1082, 1083, 1084, 1085, 1086, 1087, 1088, 1089, 1090, 1091, 1092, 1093, 1094, 1095, 1096, 1097, 1098, 1099, 1100, 1101, 1102, 1103, 1104, 1105, 1106, 1107, 1108, 1109, 1110, 1111, 1112, 1113, 1114, 1115, 1116, 1117 or 1118 consecutive nucleotides, wherein the first nucleotide deleted is the cytosine nucleotide [‘C’ ] at position 1 of SEQ ID NO:1.
2. The isolated polynucleotide of embodiment 1, wherein the polynucleotide is an embryo-specific promoter.
3. An isolated polynucleotide comprising:
(a) a nucleotide sequence comprising the sequence set forth in SEQ ID NO:1, SEQ ID NO:2, SEQ ID NO:3, SEQ ID NO:4, or SEQ ID NO:5, or SEQ ID NO:6, or a functional fragment thereof; or,
(b) a full-length complement of (a); or,
(c) a nucleotide sequence comprising a sequence having at least 90% sequence identity, based on the BLASTN method of alignment, when compared to the nucleotide sequence of (a);

wherein said nucleotide sequence is a promoter.

4. The isolated polynucleotide of embodiment 3, wherein the nucleotide sequence of (b) has at least 95% identity, based on the BLASTN method of alignment, when compared to the sequence set forth in SEQ ID NO:1.
5. The isolated polynucleotide of embodiment 3, wherein the polynucleotide is an embryo-specific promoter.
6. A recombinant DNA construct comprising the isolated polynucleotide of any one of embodiments 1-5 operably linked to at least one heterologous nucleotide sequence.
7. A vector comprising the recombinant DNA construct of embodiment 6.
8. A cell comprising the recombinant DNA construct of embodiment 6.
9. The cell of embodiment 8, wherein the cell is a plant cell.
10. A transgenic plant having stably incorporated into its genome the recombinant DNA construct of embodiment 6.
11. The transgenic plant of embodiment 10 wherein said plant is a dicot plant.
12. The transgenic plant of embodiment 11 wherein the plant is soybean.
13. A transgenic seed produced by the transgenic plant of embodiment 10.
14. The recombinant DNA construct according to embodiment 6, wherein the at least one heterologous nucleotide sequence codes for a gene selected from the group consisting of: a reporter gene, a selection marker, a disease resistance conferring gene, a herbicide resistance conferring gene, an insect resistance conferring gene; a gene involved in carbohydrate metabolism, a gene involved in fatty acid metabolism, a gene involved in amino acid metabolism, a gene involved in plant development, a gene involved in plant growth regulation, a gene involved in yield improvement, a gene involved in drought resistance, a gene involved in cold resistance, a gene involved in heat resistance and a gene involved in salt resistance in plants.
15. The recombinant DNA construct according to embodiment 6, wherein the at least one heterologous nucleotide sequence encodes a protein selected from the group consisting of: a reporter protein, a selection marker, a protein conferring disease resistance, protein conferring herbicide resistance, protein conferring insect resistance; protein involved in carbohydrate metabolism, protein involved in fatty acid metabolism, protein involved in amino acid metabolism, protein involved in plant development, protein involved in plant growth regulation, protein involved in yield improvement, protein involved in drought resistance, protein involved in cold resistance, protein involved in heat resistance and protein involved in salt resistance in plants.
16. A method of expressing a coding sequence or a functional RNA in a plant comprising:
a) introducing the recombinant DNA construct of embodiment 6 into the plant, wherein the at least one heterologous nucleotide sequence comprises a coding sequence or a functional RNA;
b) growing the plant of step a); and
c) selecting a plant displaying expression of the coding sequence or the functional RNA of the recombinant DNA construct.
   17. A method of transgenically altering a marketable plant trait, comprising:
a) introducing a recombinant DNA construct of embodiment 6 into the plant;
b) growing a fertile, mature plant resulting from step a); and
c) selecting a plant expressing the at least one heterologous nucleotide sequence in at least one plant tissue based on the altered marketable trait.
   18. The method of embodiment 17 wherein the marketable trait is selected from the group consisting of: disease resistance, herbicide resistance, insect resistance carbohydrate metabolism, fatty acid metabolism, amino acid metabolism, plant development, plant growth regulation, yield improvement, drought resistance, cold resistance, heat resistance, and salt resistance.
   19. A method for altering expression of at least one heterologous nucleotide sequence in a plant comprising:
(a) transforming a plant cell with the recombinant DNA construct of embodiment 6;
(b) growing fertile mature plants from transformed plant cell of step (a); and
(c) selecting plants containing the transformed plant cell wherein the expression of the heterologous nucleotide sequence is increased or decreased.
   20. The method of Embodiment 19 wherein the plant is a soybean plant.
   21. A method for expressing a yellow fluorescent protein ZS-GREEN1 in a host cell comprising:

(a) transforming a host cell with the recombinant DNA construct of embodiment 6; and,

(b) growing the transformed host cell under conditions that are suitable for expression of the recombinant DNA construct, wherein expression of the recombinant DNA construct results in production of increased levels of ZS-GREEN1 protein in the transformed host cell when compared to a corresponding non-transformed host cell.

22. A plant stably transformed with a recombinant DNA construct comprising a soybean embryo-specific promoter and at least one heterologous nucleotide sequence operably linked to said embryo-specific promoter, wherein said embryo-specific promoter is a capable of controlling expression of said heterologous nucleotide sequence in a plant cell, and further wherein said embryo-specific promoter comprises a fragment of SEQ ID NO:1.

### EXAMPLES

The present invention is further defined in the following Examples, in which parts and percentages are by weight and degrees are Celsius, unless otherwise stated. Sequences of promoters, cDNA, adaptors, and primers listed in this invention all are in the 5′ to 3′ orientation unless described otherwise. Techniques in molecular biology were typically performed as described in Ausubel, F. M. et al., In Current Protocols in Molecular Biology; John Wiley and Sons: New York, 1990 or Sambrook, J. et al., In Molecular Cloning: A Laboratory Manual; 2^{nd} ed.; Cold Spring Harbor Laboratory Press: Cold Spring Harbor, N.Y., 1989 (hereinafter “Sambrook et al., 1989”). It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various usages and conditions. Thus, various modifications of the invention in addition to those shown and described herein will be apparent to those skilled in the art from the foregoing description. Such modifications are also intended to fall within the scope of the appended claims.

The disclosure of each reference set forth herein is incorporated herein by reference in its entirety.

### Example 1

### Identification of Soybean Embryo-Specific Promoter Candidate Genes

Soybean expression sequence tags (EST) were generated by sequencing randomly selected clones from cDNA libraries constructed from different soybean tissues. Multiple EST sequences could often be found with different lengths representing the different regions of the same soybean gene. If more EST sequences representing the same gene are more frequently found from a tissue-specific cDNA library such as a flower library than from a leaf library, there is a possibility that the represented gene could be a flower preferred gene candidate. Likewise, if similar numbers of ESTs for the same gene were found in various libraries constructed from different tissues, the represented gene could be a constitutively expressed gene. Multiple EST sequences representing the same soybean gene could be compiled electronically based on their overlapping sequence homology into a unique full length sequence representing the gene. These assembled unique gene sequences were accumulatively collected in Pioneer Hi-Bred Int'l proprietary searchable databases.

To identify strong embryo-specific promoter candidate genes, searches were performed to look for gene sequences that were found at high frequencies in embryos and low in other tissue libraries such as leaf, root, flower, pod, etc. One unique gene PSO333255 was identified in the search to be an embryo-specific gene candidate. PSO333255 cDNA sequence (SEQ ID NO:15) as well as its putative translated protein sequence (SEQ ID NO:16) were used to search National Center for Biotechnology Information (NCBI) databases. Both PSO333255 nucleotide and amino acid sequences were found to have high homology to Bowman-Birk type proteinase isoinhibitor protein genes discovered in several plant species including soybean (NCBI accession No. AB081836.1; SEQ ID NO: 44 and NCBI accession BAB86786.1; SEQ ID NO: 45; Yoshimi et al., Biosci. Biotechnol. Biochem. 68:1279-1286 (2004)).

### Example 2

### BBI3 Gene Expression Profiles in Soybean

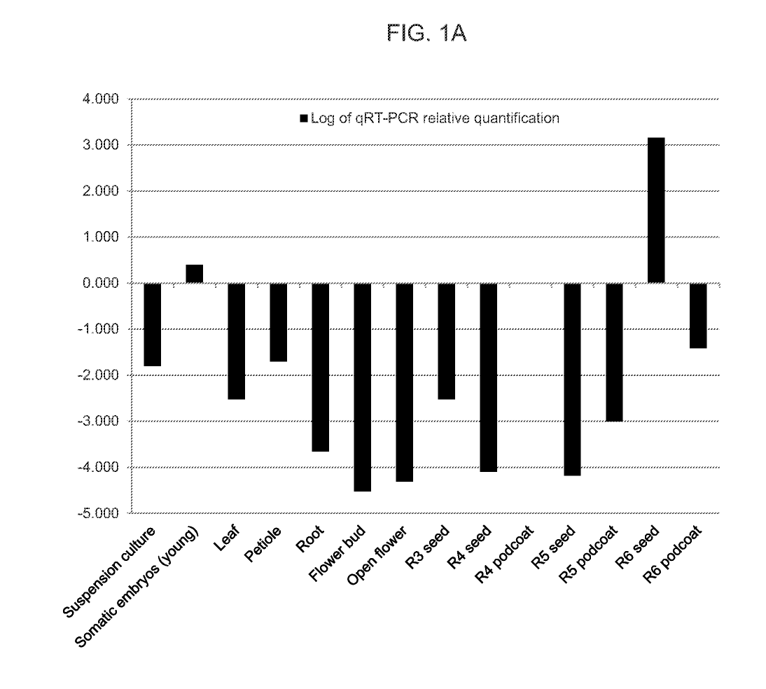

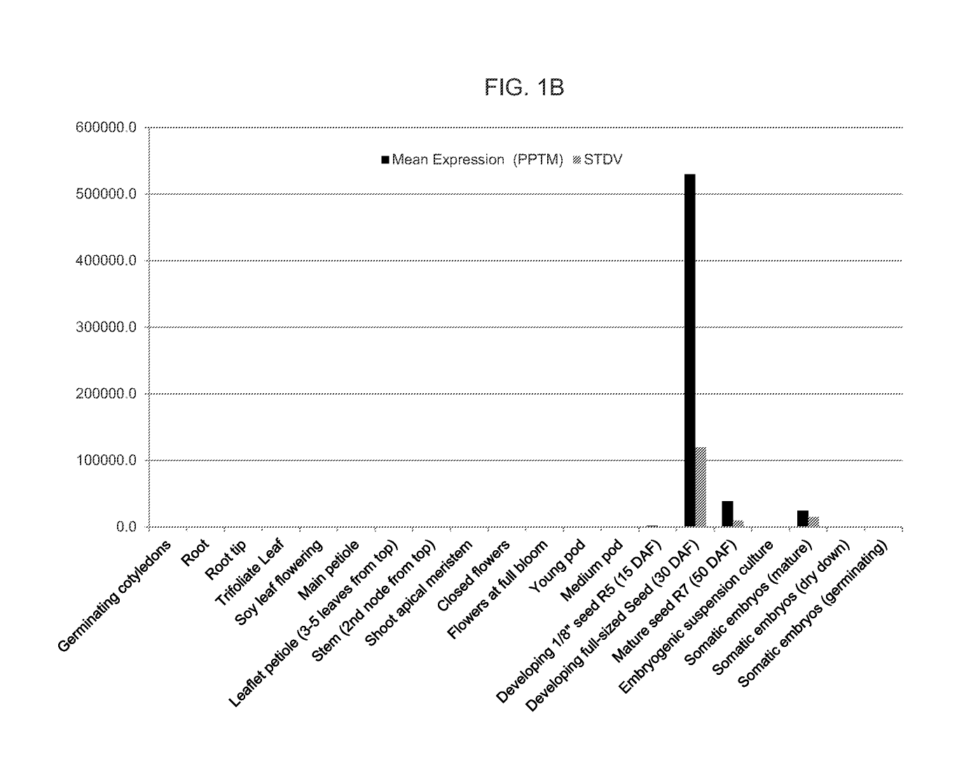

The expression profile of PSO0333255 was confirmed and extended by analyzing 14 different soybean tissues using the relative quantitative RT-PCR technique with a ABI17500 real time PCR system (Applied Biosystems, Foster City, Calif.). Fourteen soybean tissues, somatic embryo, somatic embryo one week on charcoal plate, leaf, leaf petiole, root, flower bud, open flower, R3 pod, R4 seed, R4 pod coat, R5 seed, R5 pod coat, R6 seed, R6 pod coat were collected from cultivar ‘Jack’ and flash frozen in liquid nitrogen. The seed and pod development stages were defined according to descriptions in Fehr and Caviness, IWSRBC 80:1-12 (1977). Total RNA was extracted with TRIzol® reagents (Invitrogen, Carlsbad, Calif.) and treated with DNase I to remove any trace amount of genomic DNA contamination. The first strand cDNA was synthesized using the Superscript™ III reverse transcriptase (Invitrogen). Regular PCR analysis was done to confirm that the cDNA was free of any genomic DNA using primers shown in SEQ ID NO:23 and 24. The primers are specific to the 5′UTR intron/exon junction regions of a soybean S-adenosylmethionine synthetase gene promoter SAMS (U.S. Pat. No. 7,217,858). PCR using this primer set will amplify a 967 bp DNA fragment from any soybean genomic DNA template and a 376 bp DNA fragment from the cDNA template. Genome DNA-free cDNA aliquots were used in quantitative RT-PCR analysis in which an endogenous soybean ATP sulfurylase gene (ATPS) was used as an internal control and wild type soybean genomic DNA was used as the calibrator for relative quantification. PSO33255 gene-specific primers SEQ ID NO:25 and 26 and ATPS gene-specific primers SEQ ID NO:27 and 28 were used in separate PCR reactions using the Power Sybr® Green real time PCR master mix (Applied Biosystems). PCR reaction data were captured and analyzed using the sequence detection software provided with the AB17500 real time PCR system. The logarithm values of relative quantifications of gene expression in the fourteen tissues were graphed for comparison. The qRT-PCR expression profiling of the PSO333255 BBI3 gene confirmed its strong embryo-specific expression in R6 seeds and somatic embryos (FIG. 1A). No significant expression was detected in the other tissues as indicated by the approximately −1.5 to −4.0 logarithms, i.e., approximately 30-10,000 times less abundant of the RNA transcripts relative to the DNA copies of the same gene in soybean genome.

Solexa digital gene expression dual-tag-based mRNA profiling using the Illumina (Genome Analyzer) GA2 machine is a restriction enzyme site anchored tag-based technology, in this regard similar to Mass Parallel Signature Sequence transcript profiling technique (MPSS), but with two key differences (Morrissy et al., Genome Res. 19:1825-1835 (2009); Brenner et al., Proc. Natl. Acad. Sci. USA 97:1665-70 (2000)). Firstly, not one but two restriction enzymes were used, DpnII and NlaI, the combination of which increases gene representation and helps moderate expression variances. The aggregate occurrences of all the resulting sequence reads emanating from these DpnII and NlaI sites, with some repetitive tags removed computationally, were used to determine the overall gene expression levels. Secondly, the tag read length used here is 21 nucleotides, giving the Solexa tag data higher gene match fidelity than the shorter 17-mers used in MPSS. Soybean mRNA global gene expression profiles are stored in a Pioneer proprietary database TDExpress (Tissue Development Expression Browser).

Candidate genes with different expression patterns can be searched, retrieved, and further evaluated.

The Bowman-Birk type proteinase isoinhibitor protein gene PSO333255 (BBI3) corresponds to predicted gene Glyma16g33400.1 in soybean genome, sequenced by the DOE-JGI Community Sequencing Program consortium (Schmutz J, et al., Nature 463:178-183 (2010)). The BBI3 expression profiles in twenty tissues were retrieved from the TDExpress database using the gene ID Glyma16g33400.1 and presented as parts per ten millions (PPTM) averages of three experimental repeats (FIG. 1B). The BBI3 gene expression is strongest in developed full size seeds and at relatively lower levels in mature seeds and somatic embryos, which is consistent with its EST as well as qRT-PCR expression profiles as a strong embryo-specific gene.

### Example 3

### Isolation of Soybean BBI3 Promoter

The PSO333255 cDNA sequence was BLAST searched against the soybean genome sequence database to identify corresponding genomic DNA. The −1.5 kb sequence upstream of the BBI3 start codon ATG was selected as BBI3 promoter to be amplified by PCR (polymerase chain reaction). The primers shown in SEQ ID NO:7 and 8 were then designed to amplify by PCR the putative full length 1363 bp BBI3 promoter from soybean cultivar Jack genomic DNA. SEQ ID NO:7 contains a recognition site for the restriction enzyme XmaI. SEQ ID NO:8 contains a recognition site for the restriction enzyme NcoI. The XmaI and NcoI sites were included for subsequent cloning.

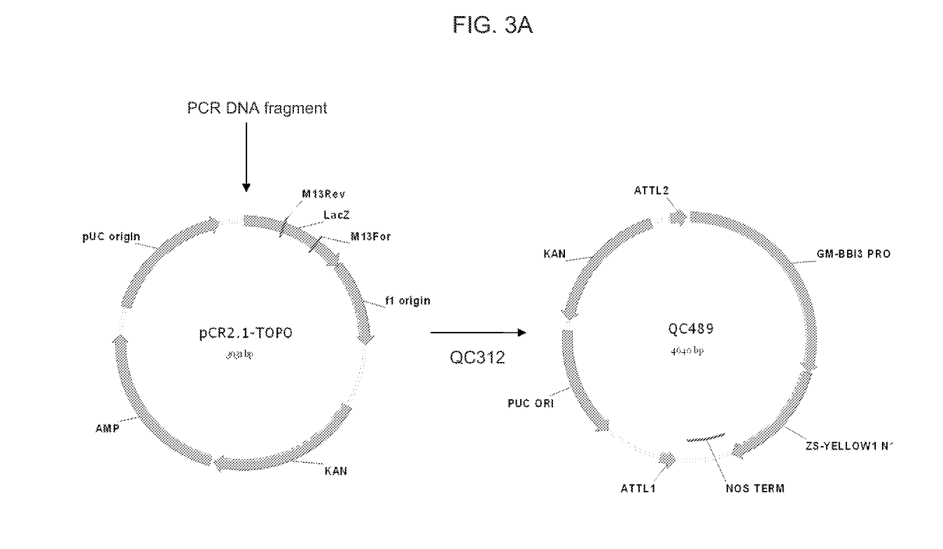

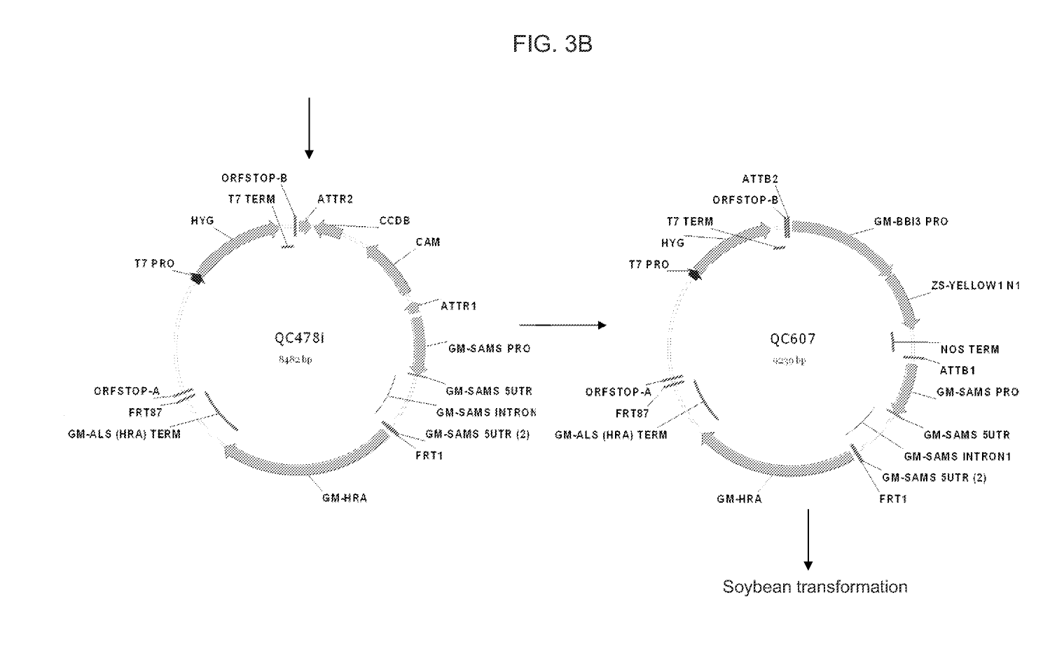

PCR cycle conditions were 94° C. for 4 minutes; 35 cycles of 94° C. for 30 seconds, 60° C. for 1 minute, and 68° C. for 2 minutes; and a final 68° C. for 5 minutes before holding at 4° C. using the Platinum high fidelity Taq DNA polymerase (Invitrogen). The PCR reaction was resolved using agarose gel electrophoresis to identify the right size PCR product representing the 1363 bp BBI3 promoter. The PCR fragment was first cloned into pCR2.1-TOPO vector by TA cloning and multiple clones were sequenced (Invitrogen). One clone with the correct BBI3 promoter sequence was selected and its plasmid DNA digested with XmaI and NcoI restriction enzymes to move the BBI3 promoter fragment upstream of the ZS-YELLOW N1 (YFP) fluorescent reporter gene in QC489 (SEQ ID NO:17; FIG. 3A). Construct QC489 contains the recombination sites AttL1 and AttL2 (SEQ ID NO:38 and 39) to qualify as a GATEWAY® cloning entry vector (Invitrogen). The 1363 bp BBI3 promoter sequence including the XmaI and NcoI sites is herein listed as SEQ ID NO:1.

### Example 4

### BBI3 Promoter Copy Number Analysis

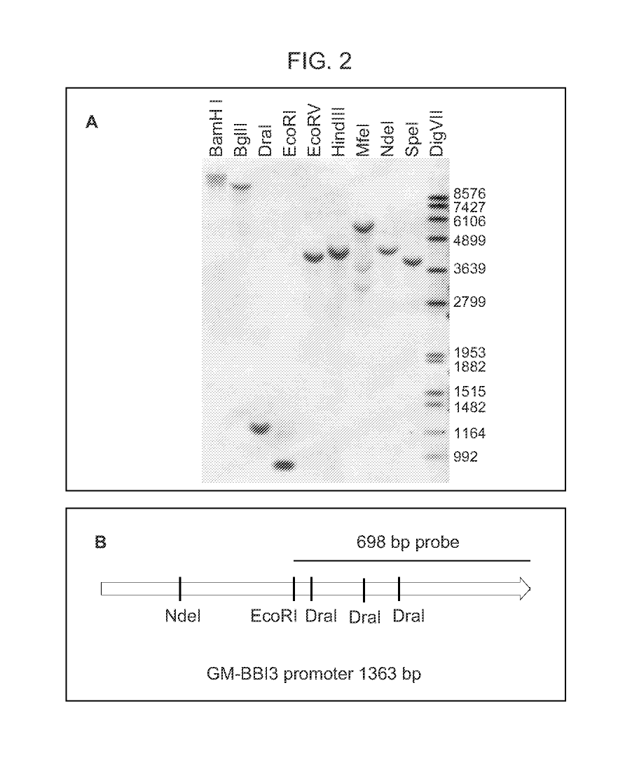

Southern hybridization analysis was performed to examine whether additional copies or sequences with significant similarity to the BBI3 promoter exist in the soybean genome. Soybean ‘Jack’ wild type genomic DNA was digested with nine different restriction enzymes, BamHI, BglII, DraI, EcoRI, EcoRV, HindIII, MfeI, NdeI, and SpeI and distributed in a 0.7% agarose gel by electrophoresis. The DNA was blotted onto Nylon membrane and hybridized at 60° C. with digoxigenin labeled BBI3 promoter DNA probe in Easy-Hyb Southern hybridization solution, and then sequentially washed 10 minutes with 2×SSC/0.1% SDS at room temperature and 3×10 minutes at 65° C. with 0.1×SSC/0.1% SDS according to the protocol provided by the manufacturer (Roche Applied Science, Indianapolis, Ind.). The BBI3 promoter probe was labeled by PCR using the DIG DNA labeling kit (Roche Applied Science) with two gene specific primers SEQ ID NO:12 and SEQ ID NO:9 to make a 698 bp long probe corresponding to the 3′ end half of the BBI3 promoter (FIG. 2B).

According to the BBI3 promoter sequence, DraI would cut the 775 bp probe region three times. The resulting fragments would be too small to be detected by Southern hybridization except for the most 3′ end 412 bp fragment that would be detected as a >412 bp band depending on the position of next downstream DraI site (FIG. 3B). None of the other eight restriction enzymes BamHI, BglII, EcoRI, EcoRV, HindIII, MfeI, NdeI, and SpeI would cut the probe region. Therefore, only one band would be expected to hybridize to the probe for each of the nine digestions if only one copy of BBI3 sequence exists in the soybean genome. The observation that only one major band was detected in all nine digestions including BamHI, BglII, DraI, EcoRI, EcoRV, HindIII, MfeI, NdeI, and SpeI suggested that there is only one copy of DNA sequence in soybean genome with significant similarity to the BBI3 promoter sequence (SEQ ID NO:1). The faint bands detected in the EcoRI and MfeI lanes suggested that there are likely other DNA sequences with low homology to the BBI3 promoter sequence in soybean genome. The sizes of the DNA molecular markers on the Southern blot are given in bp (FIG. 2A).

Since the whole soybean genome sequence is now publically available (Schmutz J, et al., Nature 463:178-183 (2010)), the BBI3 promoter copy numbers can also be evaluated by searching the soybean genome with the 1363 bp promoter sequence. Consistent with above Southern analysis, only one identical sequence Gm16:36334678-36336034 matches the BBI3 promoter sequence 1-1357. The remaining 6 bp BBI3 promoter 1358-1363 sequence CCATGGG is the NcoI site introduced artificially by the PCR primer SEQ ID NO:8. No other genomic DNA sequence was identified with significant homology to the 1363 bp sequence indicating that the BBI3 promoter is a unique sequence.

### Example 5

### BBI3:YFP Reporter Gene Constructs and Soybean Transformation

The BBI3:YFP expression cassette in GATEWAY® entry construct QC489 (SEQ ID NO:17) described in EXAMPLE 3 was moved into a GATEWAY® destination vector QC478i (SEQ ID NO:18) by LR Clonase® mediated DNA recombination between the attL1 and attL2 recombination sites (SEQ ID NO:38, and 39, respectively) in QC489 and the attR1-attR2 recombination sites (SEQ ID NO:40, and 41, respectively) in QC478i (SEQ ID NO:18; FIG. 3B). Since the destination vector QC478i already contains a soybean transformation selectable marker gene SAMS:ALS, the resulting DNA construct QC607 (SEQ ID NO:19) has two gene expression cassettes BBI3:YFP and SAMS:ALS linked together. Two 21 bp recombination sites attB1 and attB2 (SEQ ID NO:42, and 43, respectively) were newly created recombination sites resulting from DNA recombination between attL1 and attR1, and between attL2 and attR2, respectively. The 6725 bp DNA fragment containing the linked BBI3:YFP and SAMS:ALS expression cassettes was isolated from plasmid QC607 with AscI digestion, separated from the vector backbone fragment by agarose gel electrophoresis, and recovered with a DNA gel extraction kit (QIAGEN®, Valencia, Calif.). The purified DNA fragment was transformed to soybean cultivar Jack by the method of particle gun bombardment (Klein et al., Nature 327:70-73 (1987); U.S. Pat. No. 4,945,050) as described in detail below to study the BBI3 promoter activity in stably transformed soybean plants.

The same methodology as outlined above for the BBI3:YFP expression cassette construction and transformation can be used with other heterologous nucleic acid sequences encoding for example a reporter protein, a selection marker, a protein conferring disease resistance, protein conferring herbicide resistance, protein conferring insect resistance; protein involved in carbohydrate metabolism, protein involved in fatty acid metabolism, protein involved in amino acid metabolism, protein involved in plant development, protein involved in plant growth regulation, protein involved in yield improvement, protein involved in drought resistance, protein involved in cold resistance, protein involved in heat resistance and salt resistance in plants.

Soybean somatic embryos from the Jack cultivar were induced as follows. Cotyledons (˜3 mm in length) were dissected from surface sterilized, immature seeds and were cultured for 6-10 weeks in the light at 26° C. on a Murashige and Skoog media containing 0.7% agar and supplemented with 10 mg/ml 2,4-D (2,4-Dichlorophenoxyacetic acid). Globular stage somatic embryos, which produced secondary embryos, were then excised and placed into flasks containing liquid MS medium supplemented with 2,4-D (10 mg/ml) and cultured in the light on a rotary shaker. After repeated selection for clusters of somatic embryos that multiplied as early, globular staged embryos, the soybean embryogenic suspension cultures were maintained in 35 ml liquid media on a rotary shaker, 150 rpm, at 26° C. with fluorescent lights on a 16:8 hour day/night schedule. Cultures were subcultured every two weeks by inoculating approximately 35 mg of tissue into 35 ml of the same fresh liquid MS medium.

Soybean embryogenic suspension cultures were then transformed by the method of particle gun bombardment using a DuPont Biolistic™ PDS1000/HE instrument (Bio-Rad Laboratories, Hercules, Calif.). To 50 μl of a 60 mg/ml 1.0 mm gold particle suspension were added (in order): 30 μl of 30 ng/μl QC383 DNA fragment BBI3:YFP+SAMS:ALS, 20 μl of 0.1 M spermidine, and 25 μl of 5 M CaCl₂. The particle preparation was then agitated for 3 minutes, spun in a centrifuge for 10 seconds and the supernatant removed. The DNA-coated particles were then washed once in 400 μl 100% ethanol and resuspended in 45 μl of 100% ethanol. The DNA/particle suspension was sonicated three times for one second each. 5 μl of the DNA-coated gold particles was then loaded on each macro carrier disk.

Approximately 300-400 mg of a two-week-old suspension culture was placed in an empty 60×15 mm Petri dish and the residual liquid removed from the tissue with a pipette. For each transformation experiment, approximately 5 to 10 plates of tissue were bombarded. Membrane rupture pressure was set at 1100 psi and the chamber was evacuated to a vacuum of 28 inches mercury. The tissue was placed approximately 3.5 inches away from the retaining screen and bombarded once. Following bombardment, the tissue was divided in half and placed back into liquid media and cultured as described above.

Five to seven days post bombardment, the liquid media was exchanged with fresh media containing 100 ng/ml chlorsulfuron as selection agent. This selective media was refreshed weekly. Seven to eight weeks post bombardment, green, transformed tissue was observed growing from untransformed, necrotic embryogenic clusters. Isolated green tissue was removed and inoculated into individual flasks to generate new, clonally propagated, transformed embryogenic suspension cultures. Each clonally propagated culture was treated as an independent transformation event and subcultured in the same liquid MS media supplemented with 2,4-D (10 mg/ml) and 100 ng/ml chlorsulfuron selection agent to increase mass. The embryogenic suspension cultures were then transferred to agar solid MS media plates without 2,4-D supplement to allow somatic embryos to develop. A sample of each event was collected at this stage for quantitative PCR analysis.

Cotyledon stage somatic embryos were dried-down (by transferring them into an empty small Petri dish that was seated on top of a 10 cm Petri dish containing some agar gel to allow slow dry down) to mimic the last stages of soybean seed development. Dried-down embryos were placed on germination solid media and transgenic soybean plantlets were regenerated. The transgenic plants were then transferred to soil and maintained in growth chambers for seed production.

Genomic DNA were extracted from somatic embryo samples and analyzed by quantitative PCR using the 7500 real time PCR system (Applied Biosystems) with gene-specific primers and FAM-labeled fluorescence probes to check copy numbers of both the SAMS:ALS expression cassette and the BBI3:YFP expression cassette. The qPCR analysis was done in duplex reactions with a heat shock protein (HSP) gene as the endogenous controls and a transgenic DNA sample with a known single copy of SAMS:ALS or YFP transgene as the calibrator using the relative quantification methodology (Applied Biosystems). The endogenous control HSP probe was labeled with VIC and the target gene SAMS:ALS or YFP probe was labeled with FAM for the simultaneous detection of both fluorescent probes (Applied Biosystems).

The primers and probes used in the qPCR analysis are listed below.

SAMS forward primer: SEQ ID NO:29
FAM labeled SAMS probe: SEQ ID NO:30
SAMS reverse primer: SEQ ID NO:31
YFP forward primer: SEQ ID NO:32
FAM labeled YFP probe: SEQ ID NO:33
YFP reverse primer: SEQ ID NO:34
HSP forward primer: SEQ ID NO:35
VIC labeled HSP probe: SEQ ID NO:36
HSP reverse primer: SEQ ID NO:37

Only transgenic soybean events containing 1 or 2 copies of both the SAMS:ALS expression cassette and the BBI3:YFP expression cassette were selected for further gene expression evaluation and seed production (see Table 1). Events negative for YFP qPCR or with more than 2 copies for the SAMS qPCR were not further followed. YFP expressions are described in detail in EXAMPLE 8 and are also summarized in Table 1 in which the symbols “++”, “+”, and “−” indicate strong positive, positive, and negative of YFP fluorescent signals.

TABLE 1
Relative transgene copy numbers and YFP expression of
BBI3:YFP transgenic plants

| | YFP | | SAMS |
|---|---|---|---|
| Clone ID | expression | YFP qPCR | qPCR |
| 7326.1.1 | + | 1.0 | 0.7 |
| 7326.1.2 | ++ | 1.3 | 0.4 |
| 7326.2.1 | − | 0.0 | 0.6 |
| 7326.2.2 | ++ | 1.7 | 1.3 |
| 7326.3.1 | ++ | 0.8 | 0.7 |
| 7326.4.1 | + | 1.7 | 1.1 |
| 7326.5.1 | + | 1.0 | 0.3 |
| 7326.5.2 | + | 1.2 | 0.4 |
| 7326.6.1 | ++ | 1.1 | 0.4 |
| 7326.6.2 | + | 0.9 | 0.7 |
| 7326.7.1 | + | 1.2 | 0.0 |
| 7326.7.2 | ++ | 2.0 | 1.7 |
| 7326.8.1 | − | 0.1 | 0.3 |
| 7326.8.2 | + | 1.0 | 0.5 |
| 7326.9.1 | + | 1.3 | 0.3 |
| 7326.9.2 | ++ | 1.6 | 1.0 |
| 7326.10.1 | − | 0.8 | 0.6 |
| 7326.10.2 | ++ | 2.7 | 1.9 |
| 7326.12.1 | + | 1.6 | 1.2 |
| 7326,12.2 | ++ | 1.6 | 1.2 |
| 7326.12.3 | ++ | 0.8 | 0.4 |
| 7326.12.4 | + | 1.0 | 0.5 |

### Example 6

### Construction of BBI3 Promoter Deletion Constructs

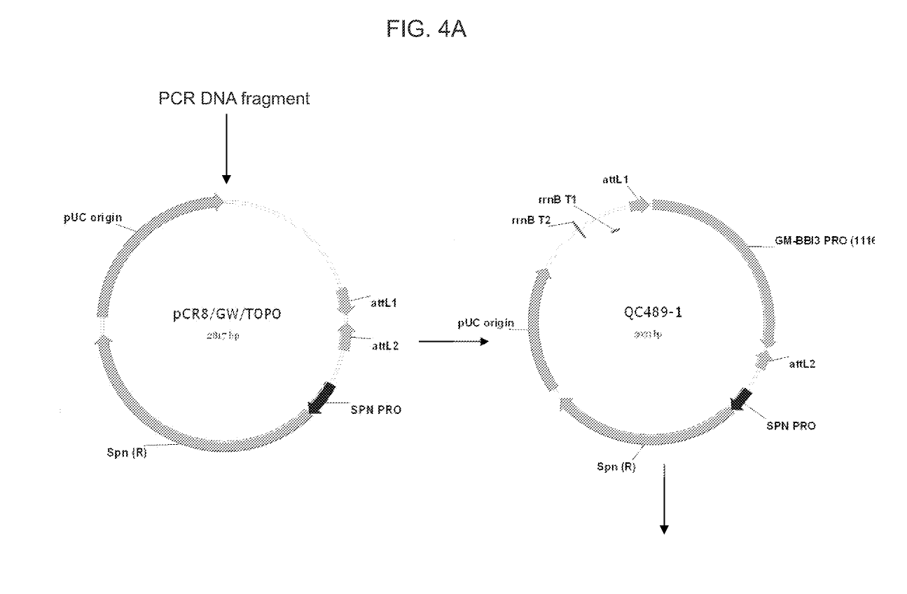

To define the transcriptional elements controlling the BBI3 promoter activity, the 1363 bp full length (SEQ ID NO:1) and five 5′ unidirectional deletion fragments 1116 bp, 885 bp, 698 bp, 473 bp, and 245 bp in length corresponding to SEQ ID NO:2, 3, 4, 5, and 6, respectively, were made by PCR amplification from the full length soybean BBI3 promoter contained in the original construct QC489 (FIG. 3A). The same antisense primer (SEQ ID NO:9) was used in the amplification by PCR of all the five BBI3 promoter truncation fragments (SEQ ID NO: 2, 3, 4, 5, and 6) by pairing with different sense primers SEQ ID NOs:10, 11, 12, 13, and 14, respectively. Each of the PCR amplified promoter DNA fragments was cloned into the GATEWAY® cloning ready TA cloning vector pCR8/GW/TOPO (Invitrogen) and clones with the correct orientation, relative to the GATEWAY® recombination sites attL1 and attL2, were selected by sequencing. The map of construct QC489-1 (SEQ ID NO:20) containing the BBI3 promoter fragment SEQ ID NO:2 is shown in FIG. 4A. The maps of constructs QC489-2, 3, 4, and 5 containing the BBI3 promoter fragments SEQ ID NOs:3, 4, 5, and 6 are similar to QC489-1 map and are not shown.

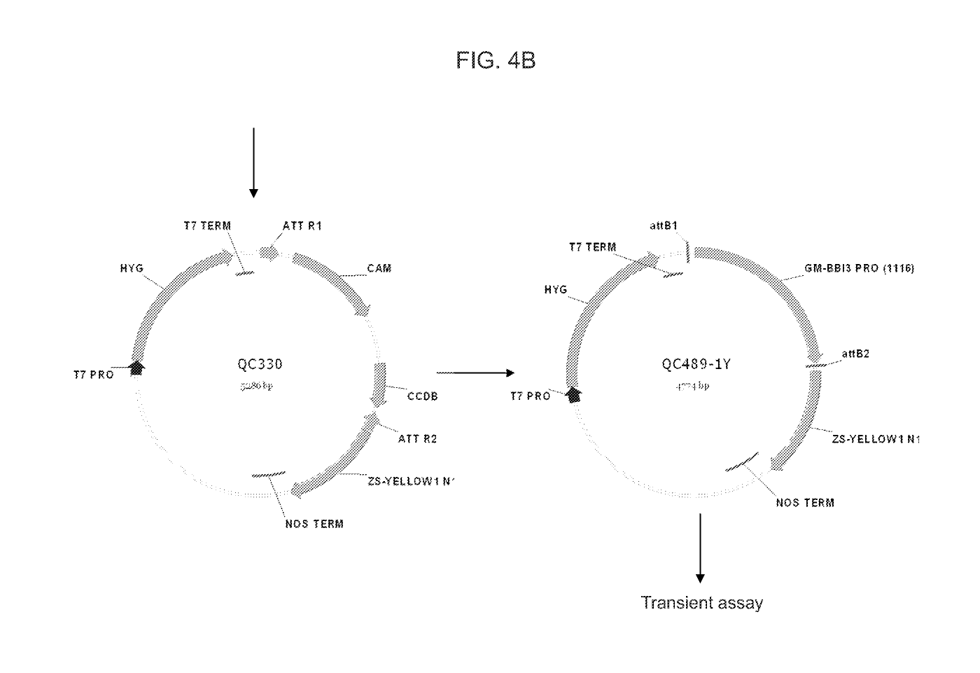

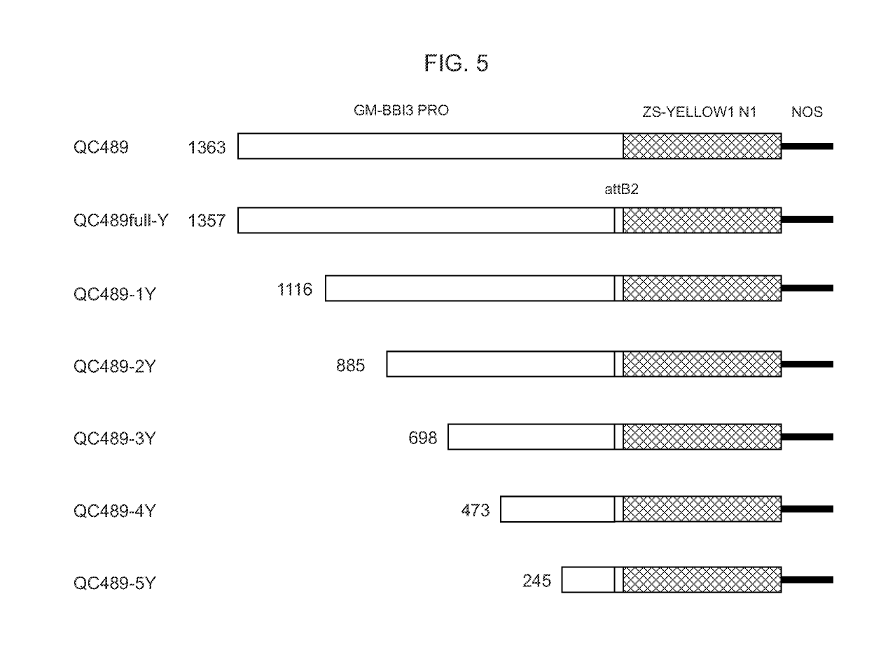

The promoter fragment in the right orientation was subsequently cloned into a GATEWAY® destination vector QC330 (SEQ ID NO:21) by GATEWAY® LR Clonase® reaction (Invitrogen) to place the promoter fragment in front of the reporter gene YFP in QC489-1Y (SEQ ID NO:22; FIG. 4B). A 21 bp GATEWAY® recombination site attB2 SEQ ID NO:43 was inserted between the promoter and the YFP reporter gene coding region as a result of the GATEWAY® cloning process. The maps of constructs QC489-2Y, 3Y, 4Y, and 5Y containing the BBI3 promoter fragments SEQ ID NOs: 3, 4, 5, and 6 are similar to QC489-1Y map and not shown. The 1357 bp near full length BBI3 promoter (with the NcoI site removed) was constructed similarly and named QC489full-Y. All the BBI3:YFP promoter deletion constructs were delivered into germinating soybean cotyledons by gene gun bombardment for transient gene expression study. The full length BBI3 promoter in QC489 that does not have the attB2 site located between the promoter and the YFP gene was included as a control. The seven BBI3 promoter fragments used in transient analysis are schematically described in FIG. 5.

### Example 7

### Transient Expression Analysis of BBI3:YFP Constructs

The constructs containing the full length and truncated BBI3 promoter fragments (QC489, QC489-fullY, QC489-1Y, 2Y, 3Y, 4Y, and 5Y) were tested by transiently expressing the ZS-YELLOW1 N1 (YFP) reporter gene in germinating soybean cotyledons. Soybean seeds were rinsed with 10% TWEEN® 20 in sterile water, surface sterilized with 70% ethanol for 2 minutes and then by 6% sodium hypochloride for 15 minutes. After rinsing the seeds were placed on wet filter paper in Petri dish to germinate for 4-6 days under light at 26° C. Green cotyledons were excised and placed inner side up on a 0.7% agar plate containing Murashige and Skoog media for particle gun bombardment. The DNA and gold particle mixtures were prepared similarly as described in EXAMPLE 5 except with more DNA (100 ng/μl). The bombardments were also carried out under similar parameters as described in EXAMPLE 5. YFP expression was checked under a Leica MZFLIII stereo microscope equipped with UV light source and appropriate light filters (Leica Microsystems Inc., Bannockburn, Ill.) and pictures were taken approximately 24 hours after bombardment with 8× magnification using a Leica DFC500 camera with settings as 0.60 gamma, 1.0× gain, 0.70 saturation, 61 color hue, 56 color saturation, and 0.51 second exposure.

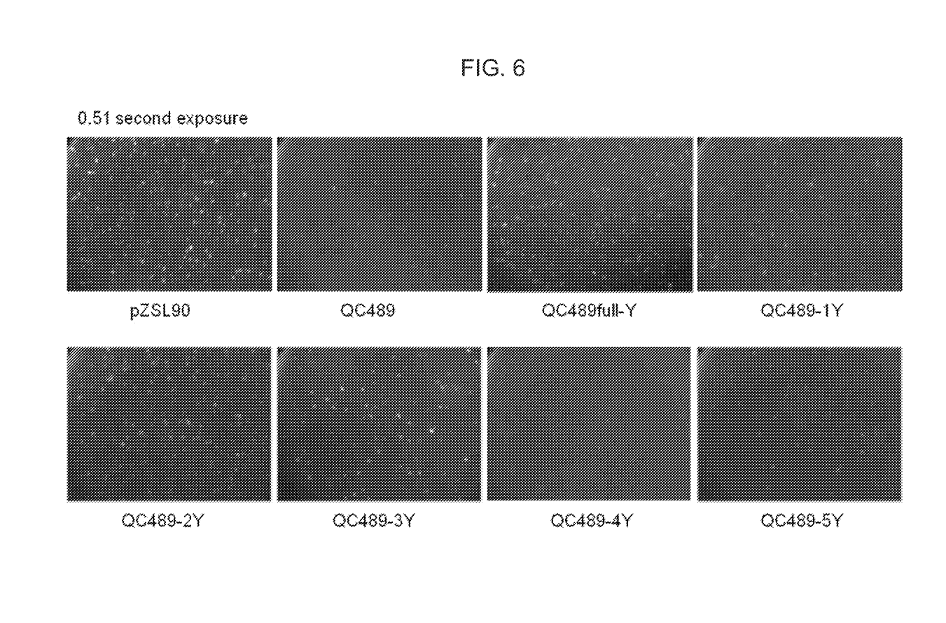

The full length BBI3 promoter construct QC489 had slightly weaker yellow fluorescence signals in transient expression assay by showing smaller yellow dots (shown as white dots in FIG. 6) than QC489full-Y which has the same full length BBI3 promoter but with the recombination site attB2 inserted between the promoter and ZS-YELLOW1 N1 coding sequences. The attB2 site did not seem to interfere negatively with promoter activity and reporter gene expression. Each bright yellow dot (shown as white dots in FIG. 6) represented a single cotyledon cell which appeared larger if the fluorescence signal was strong or smaller if the fluorescence signal was weak even under the same magnification. The full length BBI3 promoter QC489full-Y and three longer deletions constructs QC489-1Y, 2Y, and 3Y all showed similar high levels of YFP gene expression comparable to the positive control construct pZSL90 (FIG. 6). Further truncation of the BBI3 promoter to 473 bp in QC489-4Y significantly reduced its strength. The expression of the shortest construct QC489-5Y suggested that as short as 245 bp BBI3 promoter was enough to express a reporter gene thought at a reduced level. Since the expression of QC489-4Y is even weaker than the shorter QC489-5Y, there may be negative elements located in the 473-245 bp region of the BBI3 promoter (FIG. 5).

### Example 8

### BBI3:YFP Expression in Stable Transgenic Soybean Plants

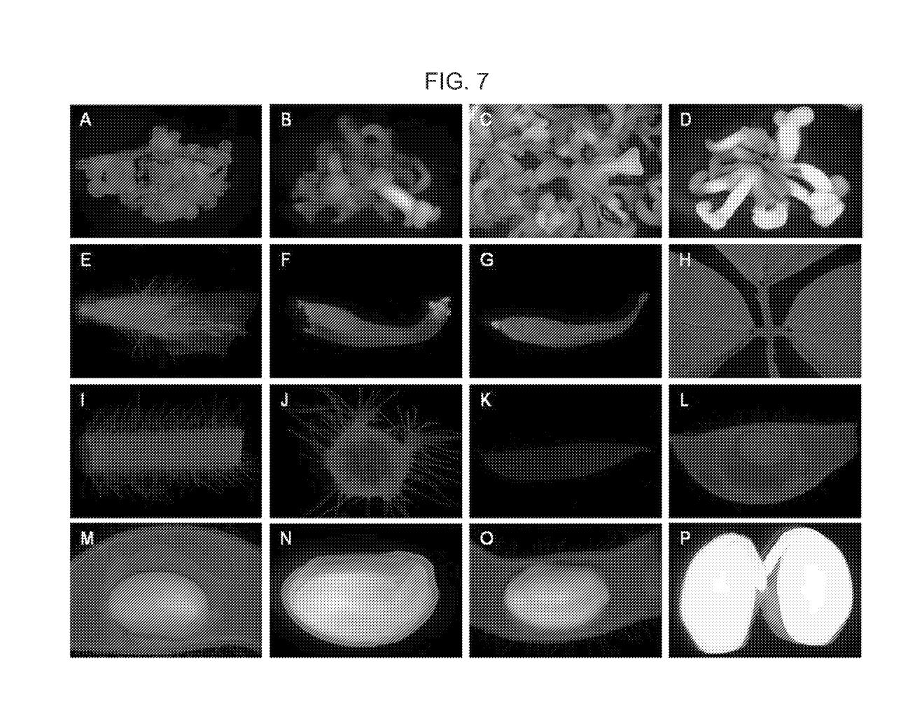

YFP gene expression was tested at different stages of transgenic plant development for yellow fluorescence emission under a Leica MZFLIII stereo microscope equipped with appropriate fluorescent light filters. Yellow fluorescence (shown as bright white areas in FIG. 7) was first detected in the cotyledons of early cotyledon embryos and throughout the somatic embryos at later stages including dried somatic embryos (FIG. 7A-D). The negative section of a positive embryo cluster emitted weak red color (shown as dark grey areas in FIG. 7A, B) due to auto fluorescence from the chlorophyll contained in soybean green tissues including embryos. Negative controls for other tissue types displayed in FIG. 7 are not shown, but any green tissue such as leaf or stem negative for YFP expression would be red and any white tissue such as root and petal would be dark yellowish under the yellow fluorescent light filter.

A soybean flower consists of five sepals, five petals including one standard large upper petal, two large side petals, and two small fused lower petals called kneel to enclose ten stamens and one pistil. The pistil consists of a stigma, a style, and an ovary in which there are 2-4 ovules. A stamen consists of a filament, and an anther on its tip. The lower majority of the nine filaments of a soybean flower are fused together to form a tube-like structure and the tenth filament is separated from the others. Pollen grains reside inside anther chambers and are released during pollination (Carlson and Lersten, In Soybeans: Improvement, Production, and Uses, 2^{nd} ed.; Wilcox et al., Eds., American Society of Agronomy, Madison, Wis., USA, 1987). Detail descriptions of soybean development stages can be found in (Fehr and Caviness, CODEN:IWSRBC 80:1-12 (1977)).

When transgenic plantlets were regenerated from somatic embryos, no yellow fluorescence signals were detected in flower including stamen and pistil (FIG. 7E-G), leaf (FIG. 7H), stem (FIG. 7I, J), or root (not shown) except auto fluorescence shown in petals, anthers, and stem trichomes. Also, no fluorescence signals were detected in developing pod coats as shown by the R3, R4, R5, and R6 pod coats in FIG. 7K-M, O, respectively.

Little yellow fluorescence signals (shown as white areas in FIG. 7) were detected in R3 or early R4 young seeds (FIG. 7L) until in more developed R5 seeds (FIG. 7M). The signals were primarily present in embryos rather than in seed coat (FIG. 7N). Strong fluorescence signals were detected in the embryos of fully developed R6 seeds (FIG. 7O). Even stronger signals were detected in drying down R7, and mature R8 seeds in which YFP proteins could also have accumulated (not shown). In conclusion, BBI3:YFP expression was detected exclusively in somatic embryos starting weakly in early cotyledon stage to the strongest in dried down somatic embryos, and similarly in zygotic seeds from weakly in early R5 to the strongest in fully developed R6, R7 seeds. BBI3 promoter thus can be used as a strong embryo-specific promoter to express genes at early cotyledon and throughout later stages of embryo development.

1-2. (canceled)

3. A recombinant DNA construct comprising:
(a) a nucleotide sequence comprising any one of the sequences set forth in SEQ ID NO:1, SEQ ID NO:2, SEQ ID NO:3, SEQ ID NO:4, SEQ ID NO:5, or SEQ ID NO:6, or,
(b) a full-length complement of (a); or,
(c) a nucleotide sequence comprising a sequence having at least 90% sequence identity, based on the BLASTN method of alignment, when compared to the nucleotide sequence of (a);
operably linked to at least one heterologous sequence,
wherein said nucleotide sequence is a promoter.

4. The recombinant DNA construct of claim 3, wherein the nucleotide sequence of (c) has at least 95% identity, based on the BLASTN method of alignment, when compared to the sequence set forth in SEQ ID NO:1.

5. The recombinant DNA construct of claim 3, wherein said promoter is an embryo-specific promoter.

6. (canceled)

7. A vector comprising the recombinant DNA construct of claim 1.

8. A cell comprising the recombinant DNA construct of claim 1.

9. The cell of claim 8, wherein the cell is a plant cell.

10. A transgenic plant having stably incorporated into its genome the recombinant DNA construct of claim 1.

11. The transgenic plant of claim 10 wherein said plant is a dicot plant.

12. The transgenic plant of claim 11 wherein the plant is soybean.

13. A transgenic seed produced by the transgenic plant of claim 10, wherein the transgenic seed comprises the recombinant DNA construct of claim 3.

14. The recombinant DNA construct according to claim 1, wherein the heterologous nucleotide sequence codes for a gene selected from the group consisting of: a reporter gene, a selection marker, a disease resistance conferring gene, a herbicide resistance conferring gene, an insect resistance conferring gene; a gene involved in carbohydrate metabolism, a gene involved in fatty acid metabolism, a gene involved in amino acid metabolism, a gene involved in plant development, a gene involved in plant growth regulation, a gene involved in yield improvement, a gene involved in drought resistance, a gene involved in cold resistance, a gene involved in heat resistance and a gene involved in salt resistance in plants.

15. (canceled)

16. A method of expressing a coding sequence or a functional RNA in a plant comprising:
a) introducing the recombinant DNA construct of claim 1 into the plant, wherein the at least one heterologous nucleotide sequence comprises a coding sequence or encodes a functional RNA;
b) growing the plant of step a); and
c) selecting a plant displaying expression of the coding sequence or the functional RNA of the recombinant DNA construct.

17. A method of transgenically altering a marketable plant trait, comprising:
a) introducing a recombinant DNA construct of claim 1 into the plant;
b) growing a fertile, mature plant resulting from step a); and
c) selecting a plant expressing the at least one heterologous nucleotide sequence in at least one plant tissue based on the altered marketable trait.

18. The method of claim 17 wherein the marketable trait is selected from the group consisting of: disease resistance, herbicide resistance, insect resistance carbohydrate metabolism, fatty acid metabolism, amino acid metabolism, plant development, plant growth regulation, yield improvement, drought resistance, cold resistance, heat resistance, and salt resistance.

19. A method for altering expression of at least one heterologous nucleotide sequence in a plant comprising:
(a) transforming a plant cell with the recombinant DNA construct of claim 1;
(b) growing fertile mature plants from transformed plant cell of step (a); and
(c) selecting plants containing the transformed plant cell wherein the expression of the heterologous nucleotide sequence is increased or decreased.

20. The method of claim 19 wherein the plant is a soybean plant.

21. A method for expressing a yellow fluorescent protein ZS-GREEN1 in a host cell comprising:
(a) transforming a host cell with the recombinant DNA construct of claim 1; and,
(b) growing the transformed host cell under conditions that are suitable for expression of the recombinant DNA construct, wherein expression of the recombinant DNA construct results in production of increased levels of ZS-GREEN1 protein in the transformed host cell when compared to a corresponding non-transformed host cell.

22. A plant stably transformed with a recombinant DNA construct comprising a soybean embryo-specific promoter and a heterologous nucleotide sequence operably linked to said embryo-specific promoter, wherein said embryo-specific promoter is a capable of controlling expression of said heterologous nucleotide sequence in a plant cell, and further wherein said embryo-specific promoter comprises any one of the sequences set forth in SEQ ID NO:1, SEQ ID NO:2, SEQ ID NO:3, SEQ ID NO:4, or SEQ ID NO:5, or SEQ ID NO:6.
